SEQUENCE LISTING (153 sequences)

SEQ ID NO: 1 - protein, 31 aa, Homo sapiens
HSDAVFTDQYTRLRKQVAAKKYLQSIKQKRY

SEQ ID NO: 2 - protein, 31 aa, Homo sapiens
  MOD_RES (1)..(1): ACETYLATION
HTDAVFTDQYTRLRKQVAAKKYLQSIKQKRY

SEQ ID NO: 3 - protein, 31 aa, Homo sapiens
HSDAVFTDQYTRLRKQMAAKKYLQSIKQKRY

SEQ ID NO: 4 - protein, 29 aa, Homo sapiens
HSDAVFTDQYTRLRKQVAAKKYLQSIKQK

SEQ ID NO: 5 - protein, 31 aa, Homo sapiens
HTEAVFTDQYTRLRKQVAAKKYLQSIKQKRY

SEQ ID NO: 6 - protein, 31 aa, Homo sapiens
HSDAVFTDQYTRLRKQLAVKKYLQDIKQGGT

SEQ ID NO: 7 - protein, 30 aa, Homo sapiens
HSDAVFTDQYTRLRKQMAAKKYLQSIKQKR

SEQ ID NO: 8 - protein, 31 aa, Homo sapiens
HSDAVFTDQYTRLRKQLAAKKYLQTIKQKRY

SEQ ID NO: 9 - protein, 31 aa, Homo sapiens
HSDAVFTDQYTRLRKQMAAKKYLQTIKQKRY

SEQ ID NO: 10 - protein, 31 aa, Homo sapiens
HSDAVFTDQYTRLRKQMAAHKYLQSIKQKRY

SEQ ID NO: 11 - protein, 31 aa, Homo sapiens
HSDAVFTDQYTRLRKQMAAKHYLQSIKQKRY

SEQ ID NO: 12 - protein, 30 aa, Homo sapiens
HSDAVFTDQYTRLRKQMAGKKYLQSIKQKR

SEQ ID NO: 13 - protein, 30 aa, Homo sapiens
HSDAVFTDQYTRLRKQMAKKKYLQSIKQKR

SEQ ID NO: 14 - protein, 30 aa, Homo sapiens
HSDAVFTDQYTRLRKQMARKKYLQSIKQKR

SEQ ID NO: 15 - protein, 30 aa, Homo sapiens
HSDAVFTDQYTRLRKQMASKKYLQSIKQKR

SEQ ID NO: 16 - protein, 30 aa, Homo sapiens
HSDAVFTDQYTRLRKQMAAKKYLQSIPQKR

SEQ ID NO: 17 - protein, 30 aa, Homo sapiens
HSDAVFTDQYTRLRKQMAAKKYLQSIQQKR

SEQ ID NO: 18 - protein, 30 aa, Homo sapiens
HSDAVFTDQYTRLRKQMAAKKYLQSIRQKR

SEQ ID NO: 19 - protein, 30 aa, Homo sapiens
HSDAVFTDQYTRLRKQMAAKKYLQSIKQRR

SEQ ID NO: 20 - protein, 30 aa, Homo sapiens
HSDAVFTDQYTRLRKQMAAKKYLQSIKQKA

SEQ ID NO: 21 - protein, 30 aa, Homo sapiens
HSDAVFTDQYTRLRKQMAAKKYLQSIKQKF

SEQ ID NO: 22 - protein, 30 aa, Homo sapiens
HSDAVFTDQYTRLRKQMAAKKYLQSIKQKH

SEQ ID NO: 23 - protein, 30 aa, Homo sapiens
HSDAVFTDQYTRLRKQMAAKKYLQSIKQKI

SEQ ID NO: 24 - protein, 30 aa, Homo sapiens
HSDAVFTDQYTRLRKQMAAKKYLQSIKQKK

SEQ ID NO: 25 - protein, 30 aa, Homo sapiens
HSDAVFTDQYTRLRKQMAAKKYLQSIKQKL

SEQ ID NO: 26 - protein, 30 aa, Homo sapiens
HSDAVFTDQYTRLRKQMAAKKYLQSIKQKM

SEQ ID NO: 27 - protein, 30 aa, Homo sapiens
HSDAVFTDQYTRLRKQMAAKKYLQSIKQKP

SEQ ID NO: 28 - protein, 30 aa, Homo sapiens
HSDAVFTDQYTRLRKQMAAKKYLQSIKQKQ

SEQ ID NO: 29 - protein, 30 aa, Homo sapiens
HSDAVFTDQYTRLRKQMAAKKYLQSIKQKS

SEQ ID NO: 30 - protein, 30 aa, Homo sapiens
HSDAVFTDQYTRLRKQMAAKKYLQSIKQKT

SEQ ID NO: 31 - protein, 30 aa, Homo sapiens
HSDAVFTDQYTRLRKQMAAKKYLQSIKQKV

SEQ ID NO: 32 - protein, 30 aa, Homo sapiens
HSDAVFTDQYTRLRKQMAAKKYLQSIKQKW

SEQ ID NO: 33 - protein, 30 aa, Homo sapiens
HSDAVFTDQYTRLRKQMAAKKYLQSIKQKY

SEQ ID NO: 34 - protein, 30 aa, Homo sapiens
HSDAVFTDQYTRLRKQMAGKKYLQSIKQRI

SEQ ID NO: 35 - protein, 30 aa, Homo sapiens
HSDAVFTDQYTRLRKQMAKKKYLQSIKQRI

SEQ ID NO: 36 - protein, 30 aa, Homo sapiens
HSDAVFTDQYTRLRKQMASKKYLQSIKQRI

SEQ ID NO: 37 - protein, 30 aa, Homo sapiens
HSDAVFTDQYTRLRKQMAAKKYLQSIPQRI

SEQ ID NO: 38 - protein, 30 aa, Homo sapiens
HSDAVFTDQYTRLRKQMASKKYLQSIRQRI

SEQ ID NO: 39 - protein, 31 aa, Homo sapiens
HSDAVFTDNYTRLRKQVAAKKYLQSIKQKRY

SEQ ID NO: 40 - protein, 31 aa, Homo sapiens
  MOD_RES (1)..(1): ACETYLATION
HTDAVFTDNYTRLRKQVAAKKYLQSIKQKRY

SEQ ID NO: 41 - protein, 31 aa, Homo sapiens
HSDAVFTDNYTRLRKQMAAKKYLQSIKQKRY

SEQ ID NO: 42 - protein, 29 aa, Homo sapiens
HSDAVFTDNYTRLRKQVAAKKYLQSIKQK

SEQ ID NO: 43 - protein, 31 aa, Homo sapiens
HTEAVFTDNYTRLRKQVAAKKYLQSIKQKRY

SEQ ID NO: 44 - protein, 31 aa, Homo sapiens
HSDAVFTDNYTRLRKQLAVKKYLQDIKQGGT

SEQ ID NO: 45 - protein, 30 aa, Homo sapiens
HSDAVFTDNYTRLRKQMAAKKYLQSIKQKR

SEQ ID NO: 46 - protein, 31 aa, Homo sapiens
HSDAVFTDNYTRLRKQLAAKKYLQTIKQKRY

SEQ ID NO: 47 - protein, 31 aa, Homo sapiens
HSDAVFTDNYTRLRKQMAAKKYLQTIKQKRY

SEQ ID NO: 48 - protein, 31 aa, Homo sapiens
HSDAVFTDNYTRLRKQMAAHKYLQSIKQKRY

SEQ ID NO: 49 - protein, 31 aa, Homo sapiens
HSDAVFTDNYTRLRKQMAAKHYLQSIKQKRY

SEQ ID NO: 50 - protein, 30 aa, Homo sapiens
HSDAVFTDNYTRLRKQMAGKKYLQSIKQKR

SEQ ID NO: 51 - protein, 30 aa, Homo sapiens
HSDAVFTDNYTRLRKQMAKKKYLQSIKQKR

SEQ ID NO: 52 - protein, 30 aa, Homo sapiens
HSDAVFTDNYTRLRKQMARKKYLQSIKQKR

SEQ ID NO: 53 - protein, 30 aa, Homo sapiens
HSDAVFTDNYTRLRKQMASKKYLQSIKQKR

SEQ ID NO: 54 - protein, 30 aa, Homo sapiens
HSDAVFTDNYTRLRKQMAAKKYLQSIPQKR

SEQ ID NO: 55 - protein, 30 aa, Homo sapiens
HSDAVFTDNYTRLRKQMAAKKYLQSIQQKR

SEQ ID NO: 56 - protein, 30 aa, Homo sapiens
HSDAVFTDNYTRLRKQMAAKKYLQSIRQKR

SEQ ID NO: 57 - protein, 30 aa, Homo sapiens
HSDAVFTDNYTRLRKQMAAKKYLQSIKQRR

SEQ ID NO: 58 - protein, 30 aa, Homo sapiens
HSDAVFTDNYTRLRKQMAAKKYLQSIKQKA

SEQ ID NO: 59 - protein, 30 aa, Homo sapiens
HSDAVFTDNYTRLRKQMAAKKYLQSIKQKF

SEQ ID NO: 60 - protein, 30 aa, Homo sapiens
HSDAVFTDNYTRLRKQMAAKKYLQSIKQKH

SEQ ID NO: 61 - protein, 30 aa, Homo sapiens
HSDAVFTDNYTRLRKQMAAKKYLQSIKQKI

SEQ ID NO: 62 - protein, 30 aa, Homo sapiens
HSDAVFTDNYTRLRKQMAAKKYLQSIKQKK

SEQ ID NO: 63 - protein, 30 aa, Homo sapiens
HSDAVFTDNYTRLRKQMAAKKYLQSIKQKL

SEQ ID NO: 64 - protein, 30 aa, Homo sapiens
HSDAVFTDNYTRLRKQMAAKKYLQSIKQKM

SEQ ID NO: 65 - protein, 30 aa, Homo sapiens
HSDAVFTDNYTRLRKQMAAKKYLQSIKQKP

SEQ ID NO: 66 - protein, 30 aa, Homo sapiens
HSDAVFTDNYTRLRKQMAAKKYLQSIKQKQ

SEQ ID NO: 67 - protein, 30 aa, Homo sapiens
HSDAVFTDNYTRLRKQMAAKKYLQSIKQKS

SEQ ID NO: 68 - protein, 30 aa, Homo sapiens
HSDAVFTDNYTRLRKQMAAKKYLQSIKQKT

SEQ ID NO: 69 - protein, 30 aa, Homo sapiens
HSDAVFTDNYTRLRKQMAAKKYLQSIKQKV

SEQ ID NO: 70 - protein, 30 aa, Homo sapiens
HSDAVFTDNYTRLRKQMAAKKYLQSIKQKW

SEQ ID NO: 71 - protein, 30 aa, Homo sapiens
HSDAVFTDNYTRLRKQMAAKKYLQSIKQKY

SEQ ID NO: 72 - protein, 30 aa, Homo sapiens
HSDAVFTDNYTRLRKQMAGKKYLQSIKQRI

SEQ ID NO: 73 - protein, 30 aa, Homo sapiens
HSDAVFTDNYTRLRKQMAKKKYLQSIKQRI

SEQ ID NO: 74 - protein, 30 aa, Homo sapiens
HSDAVFTDNYTRLRKQMASKKYLQSIKQRI

SEQ ID NO: 75 - protein, 30 aa, Homo sapiens
HSDAVFTDNYTRLRKQMAAKKYLQSIPQRI

SEQ ID NO: 76 - protein, 30 aa, Homo sapiens
HSDAVFTDNYTRLRKQMASKKYLQSIRQRI

SEQ ID NO: 77 - protein, 31 aa, Homo sapiens
HSDAVFTDQYTRLRKQVAAKKYLQSIKNKRY

SEQ ID NO: 78 - protein, 31 aa, Homo sapiens
  MOD_RES (1)..(1): ACETYLATION
HTDAVFTDQYTRLRKQVAAKKYLQSIKNKRY

SEQ ID NO: 79 - protein, 31 aa, Homo sapiens
HSDAVFTDQYTRLRKQMAAKKYLQSIKNKRY

SEQ ID NO: 80 - protein, 29 aa, Homo sapiens
HSDAVFTDQYTRLRKQVAAKKYLQSIKNK

SEQ ID NO: 81 - protein, 31 aa, Homo sapiens
HTEAVFTDQYTRLRKQVAAKKYLQSIKNKRY

SEQ ID NO: 82 - protein, 31 aa, Homo sapiens
HSDAVFTDQYTRLRKQLAVKKYLQDIKNGGT

SEQ ID NO: 83 - protein, 30 aa, Homo sapiens
HSDAVFTDQYTRLRKQMAAKKYLQSIKNKR

SEQ ID NO: 84 - protein, 31 aa, Homo sapiens
HSDAVFTDQYTRLRKQLAAKKYLQTIKNKRY

SEQ ID NO: 85 - protein, 31 aa, Homo sapiens
HSDAVFTDQYTRLRKQMAAKKYLQTIKNKRY

SEQ ID NO: 86 - protein, 31 aa, Homo sapiens
HSDAVFTDQYTRLRKQMAAHKYLQSIKNKRY

SEQ ID NO: 87 - protein, 31 aa, Homo sapiens
HSDAVFTDQYTRLRKQMAAKHYLQSIKNKRY

SEQ ID NO: 88 - protein, 30 aa, Homo sapiens
HSDAVFTDQYTRLRKQMAGKKYLQSIKNKR

SEQ ID NO: 89 - protein, 30 aa, Homo sapiens
HSDAVFTDQYTRLRKQMAKKKYLQSIKNKR

SEQ ID NO: 90 - protein, 30 aa, Homo sapiens
HSDAVFTDQYTRLRKQMARKKYLQSIKNKR

SEQ ID NO: 91 - protein, 30 aa, Homo sapiens
HSDAVFTDQYTRLRKQMASKKYLQSIKNKR

SEQ ID NO: 92 - protein, 30 aa, Homo sapiens
HSDAVFTDQYTRLRKQMAAKKYLQSIPNKR

SEQ ID NO: 93 - protein, 30 aa, Homo sapiens
HSDAVFTDQYTRLRKQMAAKKYLQSIQNKR

SEQ ID NO: 94 - protein, 30 aa, Homo sapiens
HSDAVFTDQYTRLRKQMAAKKYLQSIRNKR

SEQ ID NO: 95 - protein, 30 aa, Homo sapiens
HSDAVFTDQYTRLRKQMAAKKYLQSIKNRR

SEQ ID NO: 96 - protein, 30 aa, Homo sapiens
HSDAVFTDQYTRLRKQMAAKKYLQSIKNKA

SEQ ID NO: 97 - protein, 30 aa, Homo sapiens
HSDAVFTDQYTRLRKQMAAKKYLQSIKNKF

SEQ ID NO: 98 - protein, 30 aa, Homo sapiens
HSDAVFTDQYTRLRKQMAAKKYLQSIKNKH

SEQ ID NO: 99 - protein, 30 aa, Homo sapiens
HSDAVFTDQYTRLRKQMAAKKYLQSIKNKI

SEQ ID NO: 100 - protein, 30 aa, Homo sapiens
HSDAVFTDQYTRLRKQMAAKKYLQSIKNKK

SEQ ID NO: 101 - protein, 30 aa, Homo sapiens
HSDAVFTDQYTRLRKQMAAKKYLQSIKNKL

SEQ ID NO: 102 - protein, 30 aa, Homo sapiens
HSDAVFTDQYTRLRKQMAAKKYLQSIKNKM

SEQ ID NO: 103 - protein, 30 aa, Homo sapiens
HSDAVFTDQYTRLRKQMAAKKYLQSIKNKP

SEQ ID NO: 104 - protein, 30 aa, Homo sapiens
HSDAVFTDQYTRLRKQMAAKKYLQSIKNKQ

SEQ ID NO: 105 - protein, 30 aa, Homo sapiens
HSDAVFTDQYTRLRKQMAAKKYLQSIKNKS

SEQ ID NO: 106 - protein, 30 aa, Homo sapiens
HSDAVFTDQYTRLRKQMAAKKYLQSIKNKT

SEQ ID NO: 107 - protein, 30 aa, Homo sapiens
HSDAVFTDQYTRLRKQMAAKKYLQSIKNKV

SEQ ID NO: 108 - protein, 30 aa, Homo sapiens
HSDAVFTDQYTRLRKQMAAKKYLQSIKNKW

SEQ ID NO: 109 - protein, 30 aa, Homo sapiens
HSDAVFTDQYTRLRKQMAAKKYLQSIKNKY

SEQ ID NO: 110 - protein, 30 aa, Homo sapiens
HSDAVFTDQYTRLRKQMAGKKYLQSIKNRI

SEQ ID NO: 111 - protein, 30 aa, Homo sapiens
HSDAVFTDQYTRLRKQMAKKKYLQSIKNRI

SEQ ID NO: 112 - protein, 30 aa, Homo sapiens
HSDAVFTDQYTRLRKQMASKKYLQSIKNRI

SEQ ID NO: 113 - protein, 30 aa, Homo sapiens
HSDAVFTDQYTRLRKQMAAKKYLQSIPNRI

SEQ ID NO: 114 - protein, 30 aa, Homo sapiens
HSDAVFTDQYTRLRKQMASKKYLQSIRNRI

SEQ ID NO: 115 - protein, 32 aa, Homo sapiens
  MOD_RES (32)..(32): PEGylation
HSDAVFTDQYTRLRKQVAAKKYLQSIKQKRYC

SEQ ID NO: 116 - protein, 32 aa, Homo sapiens
  MOD_RES (1)..(1): ACETYLATION
HTDAVFTDQYTRLRKQVAAKKYLQSIKQKRYC

SEQ ID NO: 117 - protein, 32 aa, Homo sapiens
  MOD_RES (32)..(32): PEGylation
HSDAVFTDQYTRLRKQMAAKKYLQSIKQKRYC

SEQ ID NO: 118 - protein, 30 aa, Homo sapiens
  MOD_RES (30)..(30): PEGylation
HSDAVFTDQYTRLRKQVAAKKYLQSIKQKC

SEQ ID NO: 119 - protein, 32 aa, Homo sapiens
  MOD_RES (32)..(32): PEGylation
HTEAVFTDQYTRLRKQVAAKKYLQSIKQKRYC

SEQ ID NO: 120 - protein, 32 aa, Homo sapiens
  MOD_RES (32)..(32): PEGylation
HSDAVFTDQYTRLRKQLAVKKYLQDIKQGGTC

SEQ ID NO: 121 - protein, 31 aa, Homo sapiens
  MOD_RES (31)..(31): PEGylation
HSDAVFTDQYTRLRKQMAAKKYLQSIKQKRC

SEQ ID NO: 122 - protein, 32 aa, Homo sapiens
  MOD_RES (32)..(32): PEGylation
HSDAVFTDQYTRLRKQLAAKKYLQTIKQKRYC

SEQ ID NO: 123 - protein, 32 aa, Homo sapiens
  MOD_RES (32)..(32)
HSDAVFTDQYTRLRKQMAAKKYLQTIKQKRYC

SEQ ID NO: 124 - protein, 32 aa, Homo sapiens
  MOD_RES (32)..(32): PEGylation
HSDAVFTDQYTRLRKQMAAHKYLQSIKQKRYC

SEQ ID NO: 125 - protein, 32 aa, Homo sapiens
  MOD_RES (32)..(32): PEGylation
HSDAVFTDQYTRLRKQMAAKHYLQSIKQKRYC

SEQ ID NO: 126 - protein, 31 aa, Homo sapiens
  MOD_RES (31)..(31): PEGylation
HSDAVFTDQYTRLRKQMAGKKYLQSIKQKRC

SEQ ID NO: 127 - protein, 31 aa, Homo sapiens
  MOD_RES (31)..(31): PEGylation
HSDAVFTDQYTRLRKQMAKKKYLQSIKQKRC

SEQ ID NO: 128 - protein, 31 aa, Homo sapiens
  MOD_RES (31)..(31): PEGylation
HSDAVFTDQYTRLRKQMARKKYLQSIKQKRC

SEQ ID NO: 129 - protein, 31 aa, Homo sapiens
  MOD_RES (31)..(31): PEGylation
HSDAVFTDQYTRLRKQMASKKYLQSIKQKRC

SEQ ID NO: 130 - protein, 31 aa, Homo sapiens
  MOD_RES (31)..(31): PEGylation
HSDAVFTDQYTRLRKQMAAKKYLQSIPQKRC

SEQ ID NO: 131 - protein, 31 aa, Homo sapiens
  MOD_RES (31)..(31): PEGylation
HSDAVFTDQYTRLRKQMAAKKYLQSIQQKRC

SEQ ID NO: 132 - protein, 31 aa, Homo sapiens
  MOD_RES (31)..(31): PEGylation
HSDAVFTDQYTRLRKQMAAKKYLQSIRQKRC

SEQ ID NO: 133 - protein, 31 aa, Homo sapiens
  MOD_RES (31)..(31): PEGylation
HSDAVFTDQYTRLRKQMAAKKYLQSIKQRRC

SEQ ID NO: 134 - protein, 31 aa, Homo sapiens
  MOD_RES (31)..(31): PEGylation
HSDAVFTDQYTRLRKQMAAKKYLQSIKQKAC

SEQ ID NO: 135 - protein, 31 aa, Homo sapiens
  MOD_RES (31)..(31): PEGylation
HSDAVFTDQYTRLRKQMAAKKYLQSIKQKFC

SEQ ID NO: 136 - protein, 31 aa, Homo sapiens
  MOD_RES (31)..(31): PEGylation
HSDAVFTDQYTRLRKQMAAKKYLQSIKQKHC

SEQ ID NO: 137 - protein, 31 aa, Homo sapiens
  MOD_RES (31)..(31): PEGylation
HSDAVFTDQYTRLRKQMAAKKYLQSIKQKIC

SEQ ID NO: 138 - protein, 31 aa, Homo sapiens
  MOD_RES (31)..(31): PEGylation
HSDAVFTDQYTRLRKQMAAKKYLQSIKQKKC

SEQ ID NO: 139 - protein, 31 aa, Homo sapiens
  MOD_RES (31)..(31): PEGylation
HSDAVFTDQYTRLRKQMAAKKYLQSIKQKLC

SEQ ID NO: 140 - protein, 31 aa, Homo sapiens
  MOD_RES (31)..(31): PEGylation
HSDAVFTDQYTRLRKQMAAKKYLQSIKQKMC

SEQ ID NO: 141 - protein, 31 aa, Homo sapiens
  MOD_RES (31)..(31): PEGylation
HSDAVFTDQYTRLRKQMAAKKYLQSIKQKPC

SEQ ID NO: 142 - protein, 31 aa, Homo sapiens
  MOD_RES (31)..(31): PEGylation
HSDAVFTDQYTRLRKQMAAKKYLQSIKQKQC

SEQ ID NO: 143 - protein, 31 aa, Homo sapiens
  MOD_RES (31)..(31): PEGylation
HSDAVFTDQYTRLRKQMAAKKYLQSIKQKSC

SEQ ID NO: 144 - protein, 31 aa, Homo sapiens
  MOD_RES (31)..(31): PEGylation
HSDAVFTDQYTRLRKQMAAKKYLQSIKQKTC

SEQ ID NO: 145 - protein, 31 aa, Homo sapiens
  MOD_RES (31)..(31): PEGylation
HSDAVFTDQYTRLRKQMAAKKYLQSIKQKVC

SEQ ID NO: 146 - protein, 31 aa, Homo sapiens
  MOD_RES (31)..(31): PEGylation
HSDAVFTDQYTRLRKQMAAKKYLQSIKQKWC

SEQ ID NO: 147 - protein, 31 aa, Homo sapiens
  MOD_RES (31)..(31): PEGylation
HSDAVFTDQYTRLRKQMAAKKYLQSIKQKYC

SEQ ID NO: 148 - protein, 31 aa, Homo sapiens
  MOD_RES (31)..(31): PEGylation
HSDAVFTDQYTRLRKQMAGKKYLQSIKQRIC

SEQ ID NO: 149 - protein, 31 aa, Homo sapiens
  MOD_RES (31)..(31): PEGylation
HSDAVFTDQYTRLRKQMAKKKYLQSIKQRIC

SEQ ID NO: 150 - protein, 31 aa, Homo sapiens
  MOD_RES (31)..(31): PEGylation
HSDAVFTDQYTRLRKQMASKKYLQSIKQRIC

SEQ ID NO: 151 - protein, 31 aa, Homo sapiens
  MOD_RES (31)..(31): PEGylation
HSDAVFTDQYTRLRKQMAAKKYLQSIPQRIC

SEQ ID NO: 152 - protein, 31 aa, Homo sapiens
  MOD_RES (31)..(31): PEGylation
HSDAVFTDQYTRLRKQMASKKYLQSIRQRIC

SEQ ID NO: 153 - protein, 32 aa, Homo sapiens; identical to SEQ ID NO: 115
  MOD_RES (1)..(1): ACETYLATION

(19) United States
(12) Patent Application Publication
(10) Pub. No.: US 2008/0261863 A1
(43) Pub. Date: Oct. 23, 2008

(54) Pegylation of Vasoactive Intestinal Peptide (Vip) / Pituitary Adenylate Cyclase Activating Peptide (Pacap) Receptor 2 (Vpac2) Agonists and Methods of Use

(75) Inventors: James Whelan, Madison, CT (US); Clark Pan, Castro Valley, CA (US)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Pharmaceuticals Corp, West Haven, CT (US)

(21) Appl. No.: 11/632,465

(22) PCT Filed: Jun. 10, 2005
(86) PCT No.: PCT/US05/20469

§ 371 Date: Jan. 14, 2008

Related U.S. Application Data
(60) Provisional application No. 60/579,190, filed on Jun. 12, 2004.

(51) Int. Cl. A61K 38/28; C07K 14/00; C07H 21/00; C12N 15/63; C12N 5/00; A61P 3/10; C12P 21/00; C07K 1/107; A61K 38/16

(52) U.S. Cl. 514/4; 530/324; 536/23.1; 435/320.1; 435/325; 435/69.1; 530/345; 514/12

(57) ABSTRACT

This invention relates to modified Vasoactive Intestinal Peptide (VIP)/Pituitary Adenylate Cyclase Activating Peptide (PACAP) Receptor 2 (VPAC2) agonists (VPAC2 agonists) comprising a VPAC2 agonist linked to a polyethylene glycol polymer, as well as related formulations, dosages, and methods of administration thereof for therapeutic purposes. These VPAC2 agonists, compositions, and methods are useful in providing a treatment option for those individuals afflicted with a metabolic disorder such as diabetes, impaired glucose tolerance, metabolic syndrome, or prediabetic states, by inducing glucose-dependent insulin secretion in the absence of the therapeutically limiting side effect of reducing or lowering blood pressure.

This application claims benefit of U.S. Provisional Application Ser. No. 60/579,190; filed on Jun. 12, 2004, the contents of which are incorporated herein by reference in their entirety.

### FIELD OF THE INVENTION

This invention relates to modified Vasoactive Intestinal Peptide (VIP)/Pituitary Adenylate Cyclase Activating Peptide (PACAP) Receptor 2 (VPAC2) agonists (VPAC2 agonists) comprising a VPAC2 agonist linked to a polyethylene glycol polymer, as well as related formulations, dosages, and methods of administration thereof for therapeutic purposes. These VPAC2 agonists, compositions, and methods are useful in providing a treatment option for those individuals afflicted with a metabolic disorder such as diabetes, impaired glucose tolerance, metabolic syndrome, or prediabetic states, by inducing glucose-dependent insulin secretion in the absence of the therapeutically limiting side effect of reducing or lowering blood pressure.

### BACKGROUND OF THE INVENTION

Diabetes is characterized by impaired glucose metabolism manifesting itself, among other things, by an elevated blood glucose level in the diabetic patient. Underlying defects lead to a classification of diabetes into two major groups: type 1 diabetes, or insulin dependent diabetes mellitus (IDDM), which arises when patients lack β-cells producing insulin in their pancreatic glands, and type 2 diabetes, or non-insulin dependent diabetes mellitus (NIDDM), which occurs in patients with an impaired β-cell function and alterations in insulin action.

Type 1 diabetic patients are currently treated with insulin, while the majority of type 2 diabetic patients are treated with agents that stimulate β-cell function or with agents that enhance the tissue sensitivity of the patients towards insulin. Over time almost one-half of type 2 diabetic subjects lose their response to these agents and then must be placed on insulin therapy.

Because of the problems with current treatments, new therapies to treat type 2 diabetes are needed. In particular, new treatments to retain normal (glucose-dependent) insulin secretion are needed. Such new drugs may have the following characteristics: dependent on glucose for promoting insulin secretion (i.e., produce insulin secretion only in the presence of elevated blood glucose); low primary and secondary failure rates; and preserve islet cell function. The strategy to develop the new therapy disclosed herein is based on the cyclic adenosine monophosphate (cAMP) signaling mechanism and its effects on insulin secretion.

Cyclic AMP is a major regulator of the insulin secretion process. Elevation of this signaling molecule promotes the closure of the K+ channels following the activation of the protein kinase A pathway. Closure of the K+ channels causes cell depolarization and subsequent opening of Ca⁺⁺ channels, which in turn leads to exocytosis of insulin granules. Little if any effects on insulin secretion occurs in the presence of low glucose concentrations (Weinhaus, et al., Diabetes 47:1426-1435, 1998). Secretagogues like pituitary adenylate cyclase activating peptide (“PACAP”) and GLP-1 (glucagon-like peptide 1) use the cAMP system to regulate insulin secretion in a glucose-dependent fashion (Filipsson, et al., Diabetes 50:1959-1969, 2001; Komatsu, et al., Diabetes 46:1928-1938,1997; Drucker, Endocrinol. 142:521-527, 2001). Insulin secretagogues, such as GLP-1 and PACAP, working through the elevation of cAMP are also able to enhance insulin synthesis in addition to insulin release (Borboni, et al., Endocrinol. 140:5530-5537,1999; Skoglund, et al., Diabetes 49:5530-5537, 2000).

PACAP is a potent stimulator of glucose-dependent insulin secretion from pancreatic β-cells. Three different PACAP receptor types (PAC1, VPAC1, and VPAC2) have been described (Vaudry, et al., Pharmacol. Rev. 52:269-324, 2000; Harmar, et al., Pharmacol. Rev. 50:265-270, 1998). PACAP displays no receptor selectivities, having comparable activities and potencies at all three receptors. PAC1 is located predominately in the CNS, whereas VPAC1 and VPAC2 are more widely distributed. VPAC1 is located in the CNS as well as in liver, lungs, and intestine. VPAC2 is located in the CNS, pancreas, skeletal muscle, heart, kidney, adipose tissue, testis, and stomach. Recent work demonstrates that VPAC2 plays a role in insulin secretion from β-cells (Inagaki, et al., Proc. Natl. Acad. Sci. USA 91:2679-2683, 1994; Tsutsumi, et al., Diabetes 51:1453-1460, 2002). VPAC2 activation leads to elevation of intracellular cAMP which in turn activates the nonselective cation channels in β-cells increasing [Ca++], and promotes exocytosis of insulin-containing secretory granules.

PACAP is the newest member of the superfamily of metabolic, neuroendocrine, and neurotransmitter peptide hormones that exert their action through the cAMP-mediated signal transduction pathway (Arimura, Regul. Peptide 37:287-303, 1992). The biologically active peptides are released from the biosynthetic precursor in two molecular forms, either as a 38-amino acid peptide (PACAP-38) and/or as a 27-amino acid peptide (PACAP-27) with an amidated carboxyl termini.

The highest concentrations of the two forms of the peptide are found in the brain and testis. The shorter form of the peptide, PACAP-27, shows 68% structural homology to vasoactive intestinal polypeptide (VIP). Recent studies have demonstrated diverse biological effects of PACAP, from a role in reproduction to an ability to stimulate insulin secretion (McArdle, Endocrinol. 135:815-817,1994; Yada, et al., J. Biol. Chem. 269:1290-1293, 1994). In addition, PACAP appears to play a role in hormonal regulation of lipid and carbohydrate metabolism, circadian function, the immune system, growth, energy homeostasis, male reproductive function, regulation of appetite, as well as acute and chronic inflammatory diseases, septic shock, and autoimmune diseases (e.g., systemic lupus erythematosus) (Gray, et al., Mol. Endocrinol. 15:1739-1747, 2001; Harmar, et al., Cell 109:497-508, 2002; Asnicar, et al., Endocrinol. 143:3994-4006, 2002; Tachibana, et al., Neurosci. Lett. 339:203-206, 2003; Pozo, Trend. Mol. Med. 9:211-217, 2003).

PACAP-27 causes peripheral vasodilation that elicits a compensatory increase in heart rate (Gardiner, et al., Br. J. Pharmacol. 111:589-597,1994; Champion, et al., Ann. NY Acad. Sci. 805:429-441, 1996). To decipher which receptor mediates this cardiovascular side effect, the non-selective agonist PACAP-27, the VPAC1/VPAC2 selective agonist VIP, the PAC1-selective agonist maxadilian, the VPAC1 selective agonist PG 97-269, and the VPAC2 selective agonist BAY 55-9837 were tested for their effects on heart rate and blood pressure (Moro, et al., J. Biol. Chem. 272:966-970,1997; Gourlet, et al., Peptides 18:1555-1560,1997; Tsutsumi, et al., 2002). PACAP-27 and the PAC1 selective agonist increased the heart rate in dogs by two-fold when the peptides were injected intravenously at 0.1 nmol/kg, whereas VIP and the VPAC1-selective agonist increased heart rate by only 10-20%. The VPAC2 selective agonist had no effect at the same dose. Although most of the cardiovascular side effects could be attributed to PAC1 activation, the VPAC2 agonist still displays a significant cardiovascular effect in a murine model. It decreases mean arterial pressure in a dose-dependent fashion with an ED₅₀ of 400 pmol/kg after a single bolus intravenous injection in rats. This peptide causes a dose-dependent increase in plasma insulin levels in fasted rats with an ED₅₀ value of 3 pmol/kg by intravenous injection. Even though there appears to be a significant separation between efficacy to promote insulin secretion and the cardiovascular side effects, greater separation is required for safety in the treatment of type 2 diabetes.

Thus, the present invention provides VPAC2 agonists, compositions, and methods useful in providing a treatment option for those individuals afflicted with a metabolic disorder such as diabetes, impaired glucose tolerance, metabolic syndrome, or prediabetic states, by inducing glucose-dependent insulin secretion in the absence of the therapeutically limiting side effect of reducing or lowering blood pressure.

### SUMMARY OF THE INVENTION

This invention relates to modified VPAC2 agonists comprising a VPAC2 agonist linked to a polyethylene glycol (PEG) polymer having a molecular weight of greater than 22 kD, and which retains its ability to agonize the VPAC2. These modified VPAC2 agonists are effective in the treatment of metabolic disorders, such as diabetes or impaired glucose tolerance, a prediabetic state. Moreover, the modified VPAC2 agonists of this invention are capable of treating metabolic disorders without lowering of mean arterial pressure, thereby producing no cardiovascular side effects (such as lowering blood pressure and increase in heart rate) and thus, allowing higher more effective doses to be administered.

The polypeptides of the present invention provide a new therapy for patients with, for example, metabolic disorders such as those resulting from decreased endogenous insulin secretion, in particular type 2 diabetics, or for patients with impaired glucose tolerance, a prediabetic state that has a mild alteration in insulin secretion. In addition, the polypeptides of the present invention may be useful in the prevention and/or treatment of type 1 diabetes, gestational diabetes, maturity-onset diabetes of the young (MODY), latent autoimmune diabetes adult (LADA), and associated diabetic dyslipidemia and other diabetic complications, as well as hyperglycemia, hyperinsulinemia, impaired glucose tolerance, impaired fasting glucose, dyslipidemia, hypertriglyceridemia, Syndrome X, and insulin resistance.

One aspect of the invention is a polypeptide selected from the group consisting of SEQ ID NOs: 1 through 153, and fragments, derivatives, and variants thereof that demonstrate at least one biological function that is substantially the same as the polypeptides of the listed SEQ ID NOs. (collectively, “polypeptides of this invention”), including functional equivalents thereof.

Another embodiment of the invention is a polypeptide that encodes the polypeptides of the present invention, and the attendant vectors and host cells necessary to recombinantly express the polypeptides of this invention.

The invention is also directed to a method of treating diabetes, diabetes-related disorders, and/or other diseases or conditions affected by the polypeptides of this invention, preferably effected by the VPAC2 agonist function of the polypeptides of this invention, in a mammal, comprising administering a therapeutically effective amount of any of the polypeptides of the present invention or any polypeptide active at VPAC2.

### BRIEF DESCRIPTION OF THE DRAWING

FIGS. 1a-1d depict amino acid sequences of polypeptides of SEQ ID NOs: 1 through 153. SEQ ID NOs: 115-153 refer to peptides that are PEGylated at the C-terminal cysteine via a maleimide linkage. The PEG may be, for example, a 22 kD linear PEG or a 43 kD branched PEG.

FIG. 2 is a line graph demonstrating that the non-PEGylated peptide, SEQ ID NO:1, significantly lowers blood pressure. Animals were treated as described in the Examples. The results are expressed as a percentage of the mean blood pressure of the vehicle-treated rats.

FIG. 3 demonstrates that the peptide PEGylated with a linear 22 kD PEG, SEQ ID NO:1+PEG (22 kD), lowers blood pressure at intravenous doses of >160 pg/kg given as a bolus injection, although the blood pressure lowering effect is less than that of the non-PEGylated peptide (SEQ ID NO: 1). Animals were treated as described in the Examples.

FIG. 4 demonstrates that the peptide PEGylated with a branched 43 kD PEG, SEQ ID NO:1+PEG (43 kD), had no effect on blood pressure at intravenous doses of 1.6 to 480 μg/kg given as a bolus injection. The estimated plasma concentration following a 480 μg/kg iv dose of SEQ ID NO:1+PEG (43 kD) (>4000 nM) is estimated to be >4000-fold the plasma concentration of SEQ ID NO:1+PEG (43 kD) at the ED₅₀ in the rat IPGTT (<1 nM). Animals were treated as described in the Examples.

FIG. 5 is a bar chart illustrating insulin secretion of dispersed rat islet cells following exposure to a PEGylated peptide of the present invention.

### DETAILED DESCRIPTION OF THE INVENTION

The polypeptides of the present invention play a role in glucose homeostatsis, and in particular, these peptides function as VPAC2 agonists by lowering plasma glucose concentrations. Given VPAC2's role in promoting glucose-regulated insulin secretion in the pancreas, VPAC2 agonists are potentially valuable in the treatment of metabolic disorders and other diseases. To date, however, VPAC2 agonists have had significant side effects; namely a reduction in mean arterial pressure, which in turn can lead to an increase in heart rate, an especially dangerous condition for a type 2 diabetic.

Peptides having VPAC2 agonist activity have been identified, and include, for example, PACAP, VIP, BAY 55-9837, Ro 25-1553, Ro 25-1392, and other PACAP and VIP analogs (Gourlet, et al., Peptides 18:403-408; Xia, et al., J. Pharmacol. Exp. Ther. 281:629-633, 1997).

The inventors herein have found that modifying the polypeptides of the present invention (VPAC2 agonists) by linking a polyethylene glycol (PEG) polymer having a molecular weight of greater than 22 kD will inhibit the reduction in mean arterial pressure associated with VPAC2 agonists. Without being bound to theory, the inventors herein believe that increasing the size of a VPAC2 agonist using PEGylation technology limits VPAC2 agonist access to the less vascularized smooth muscle tissue surrounding the blood vessel wall, but not its ability to enter the highly vascularized panreatic islets. As a consequence, the PEGylated VPAC2 agonist is unable to promote vascular smooth muscle relaxation which leads to reduced blood pressure. The PEGylated VPAC2 agonist, however, still has access to the pancreas and thus, lowers blood glucose, the desired activity for treating type 2 diabetes.

Thus, this invention relates to modified VPAC2 agonists comprising a VPAC2 agonist linked to a polyethylene glycol polymer having a molecular weight of greater than 22 kD, and methods of administration thereof for therapeutic purposes are provided. These modified VPAC2 receptor agonists and compositions function in vivo as VPAC2 receptor agonists in the prevention and/or treatment of such diseases or conditions as diabetes, hyperglycemia, impaired glucose tolerance, impaired fasting glucose and obesity by inducing glucose-dependent insulin secretion, without reducing mean arterial pressure.

The polypeptides of this invention function in vivo as VPAC2 agonists or otherwise in the prevention and/or treatment of such diseases or conditions as diabetes including both type 1 and type 2 diabetes, gestational diabetes, maturity-onset diabetes of the young (MODY) (Herman, et al., Diabetes 43:40, 1994); latent autoimmune diabetes adult (LADA) (Zimmet, et al., Diabetes Med. 11:299, 1994); and associated diabetic dyslipidemia and other diabetic complications, as well as hyperglycemia, hyperinsulinemia, impaired glucose tolerance, impaired fasting glucose, dyslipidemia, hypertriglyceridemia, Syndrome X, and insulin resistance.

In one embodiment, the polypeptides of this invention stimulate insulin release from pancreatic β-cells in a glucose-dependent fashion, and the polypeptides of this invention are stable in both aqueous and non-aqueous formulations and exhibit a plasma half-life of greater than one hour.

In another embodiment, the polypeptides of this invention are selective VPAC2 agonists with greater selectivity for VPAC2 over VPAC1 and/or PAC1. The polypeptides of the present invention stimulate insulin release into plasma in a glucose-dependent fashion without inducing a stasis or increase in the level of plasma glucose that is counterproductive to the treatment of, for example, type 2 diabetes. Additionally, it is preferable for the polypeptides of this invention to be selective agonists of the VPAC2 receptor, thereby causing, for example, an increase in insulin release into plasma, while being selective against other receptors that are responsible for such side effects as gastrointestinal water retention, and/or unwanted cardiovascular effects such as reduced mean arterial pressure and increased heart rate.

Certain terms used throughout this specification are defined below. The single letter abbreviation for a particular amino acid, its corresponding amino acid, and three letter abbreviation are as follows: A, alanine (ala); C, cysteine (cys); D, aspartic acid (asp); E, glutamic acid (glu); F, phenylalanine (phe); G, glycine (gly); H, histidine (his); I, isoleucine (ile); K, lycine (lys); L, leucine (leu); M, methionine (met); N, asparagine (asn); P, proline (pro); Q, glutamine (gin); R, arginine (arg); S, serine (ser); T, threonine (thr); V, valine (val); W, tryptophan (trp); Y, tyrosine (tyr).

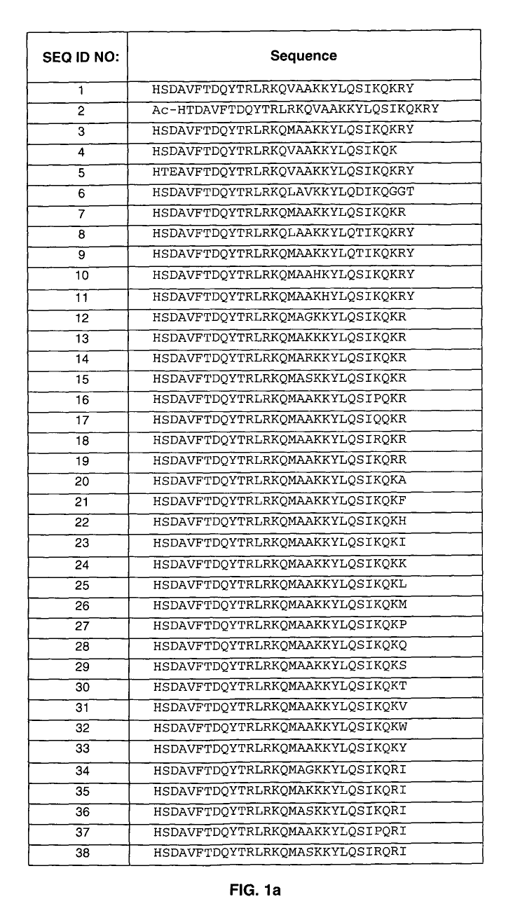

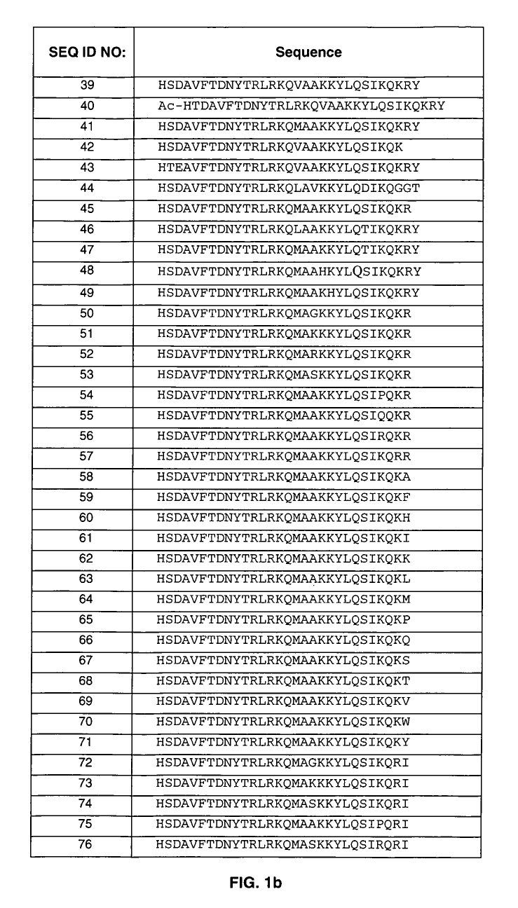

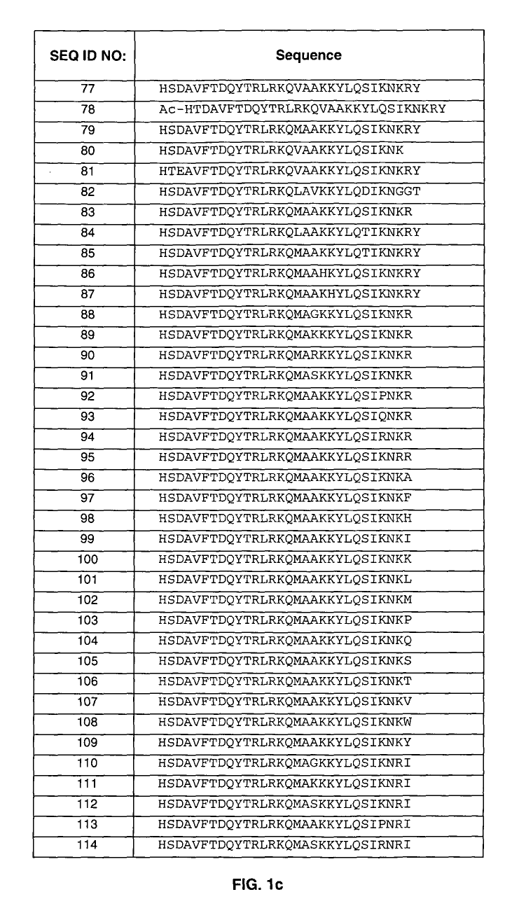

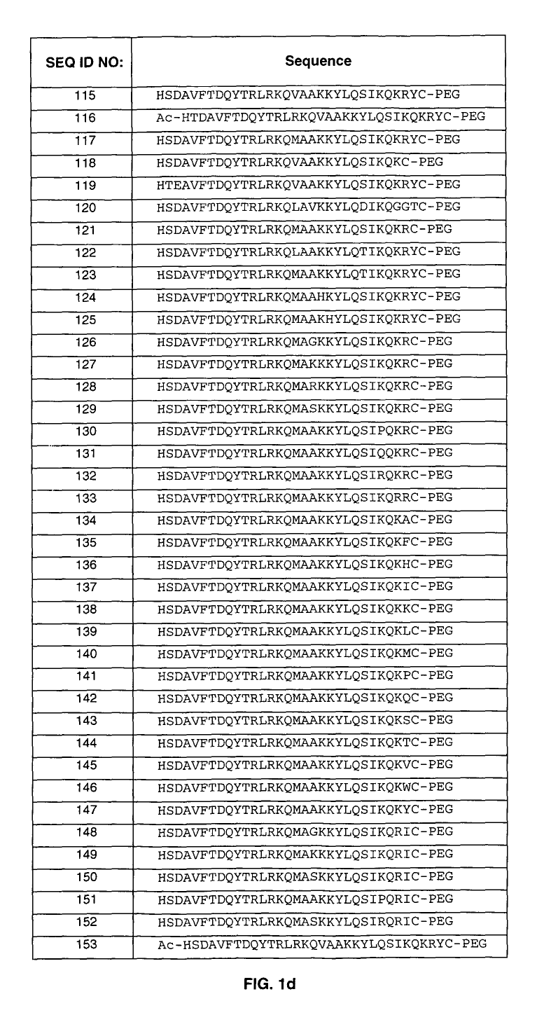

“Functional equivalent” and “substantially the same biological function or activity” each means that degree of biological activity that is within about 30% to about 100% or more of that biological activity demonstrated by the polypeptide to which it is being compared when the biological activity of each polypeptide is determined by the same procedure. For example, a polypeptide that is functionally equivalent to a polypeptide of FIG. 1 is one that, when tested in the cyclic AMP (cAMP) scintillation proximity assay described in the Examples, demonstrates accumulation of cAMP in CHO cell line expressing the human VPAC2 receptor.

The terms “fragment,” “derivative,” and “variant,” when referring to the polypeptides of FIG. 1, means fragments, derivatives, and variants of the polypeptides which retain substantially the same biological function or activity as such polypeptides, as described further below.

An analog includes a pro-polypeptide which includes within it, the amino acid sequence of the polypeptide of this invention. The active polypeptide of this invention can be cleaved from the additional amino acids that complete the pro-polypeptide molecule by natural, in vivo processes or by procedures well known in the art such as by enzymatic or chemical cleavage.

A fragment is a portion of the polypeptide which retains substantially similar functional activity, as described in the in vivo models disclosed herein.

A derivative includes all modifications to the polypeptide which substantially preserve the functions disclosed herein and include additional structure and attendant function (e.g., PEGylated polypeptides which have greater half-life), fusion polypeptides which confer targeting specificity, or an additional activity such as toxicity to an intended target.

The polypeptides of the present invention may be recombinant polypeptides, natural purified polypeptides, or synthetic polypeptides.

The fragment, derivative, or variant of the polypeptides of the present invention may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (e.g., polyethyleneglycol), or (iv) one in which the additional amino acids are fused to the mature polypeptide, such as a leader or secretory sequence or a sequence which is employed for purification of the mature polypeptide or a propolypeptide sequence, or (v) one in which the polypeptide sequence is fused with a larger polypeptide (e.g., human albumin, an antibody or Fc, for increased duration of effect). Such fragments, derivatives, and variants and analogs are deemed to be within the scope of those skilled in the art from the teachings herein.

The derivatives of the present invention may contain conservative amino acid substitutions (defined further below) made at one or more predicted, preferably nonessential amino acid residues. A “nonessential” amino acid residue is a residue that can be altered from the wild-type sequence of a protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Non-conservative substitutions would not be made for conserved amino acid residues or for amino acid residues residing within a conserved protein domain, such as residues 19 and 27 where such residues are essential for protein activity such as VPAC2 activity and/or VPAC2 selectivity. Fragments, or biologically active portions include polypeptide fragments suitable for use as a medicament, to generate antibodies, as a research reagent, and the like. Fragments include peptides comprising amino acid sequences sufficiently similar to or derived from the amino acid sequences of a polypeptide of this invention and exhibiting at least one activity of that polypeptide, but which include fewer amino acids than the full-length polypeptides disclosed herein. Typically, biologically active portions comprise a domain or motif with at least one activity of the polypeptide. A biologically active portion of a polypeptide can be a peptide which is, for example, five or more amino acids in length. Such biologically active portions can be prepared synthetically or by recombinant techniques and can be evaluated for one or more of the functional activities of a polypeptide of this invention by means disclosed herein and/or well known in the art.

Variants include polypeptides that differ in amino acid sequence due to mutagenesis. Variants that function as VPAC2 agonists can be identified by screening combinatorial libraries of mutants, for example truncation mutants, of the polypeptides of this invention for VPAC2 agonist activity.

The invention also provides chimeric or fusion polypeptides. The targeting sequence is designed to localize the delivery of the polypeptide to the pancreas to minimize potential side effects. The polypeptides of this invention can be composed of amino acids joined to each other by peptide bonds or modified peptide bonds (i.e., peptide isosteres), and may contain amino acids other than the 20 gene-encoded amino acids. The polypeptides may be modified by either natural processes, such as postranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, PEGylation, proteolytic processing, phosphorylation, prenylation, racemization, selenoylation, sulfation, transfer-RNA mediated addition of amino acids to proteins such as arginylation, and ubiquitination (see, e.g., Proteins, Structure and Molecular Properties, 2nd ed., T. E. Creighton, W. H. Freeman and Company, New York (1993); Posttranslational Covalent Modification of Proteins, B. C. Johnson, ed., Academic Press, New York, pgs. 1-12 (1983); Seifter, et al., Meth. Enzymol 182:626-646, 1990; Rattan, et al., Ann. N.Y. Acad. Sci. 663:48-62,1992).

In the case of PEGylation, the fusion of the peptide to PEG may be accomplished by any means known to one skilled in the art. For example, PEGylation may be accomplished by first introducing a cysteine mutation into the peptide to provide a linker upon which to attach the PEG, followed by site-specific derivatization with PEG-maleimide. Alternatively, the N-terminal modification may incorporate a reactive moiety for coupling to PEG, as exemplified by the amine group, the mercapto group, or the carboxylate group of the N-terminal modifying compounds disclosed above. For example, PEGylation may be accomplished by first introducing a mercapto moiety into the polypeptide via the N-terminal modifying group to provide a linker upon which to attach the PEG, followed by site-specific derivatization with methoxy-PEG-maleimide reagents supplied by, for example, either Nektar Therapeutics (San Carlos, Calif., USA) and/or NOF (Tokyo, Japan). In addition to maleimide, numerous Cys reactive groups are known to those skilled in the art of protein cross-linking, such as the use of alkyl halides and vinyl sulfones (see, e.g., Proteins, Structure and Molecular Properties, 2nd ed., T. E. Creighton, W. H. Freeman and Company, New York, 1993). In addition, the PEG could be introduced by direct attachment to the C-terminal carboxylate group, or to an internal amino acid such as Cys, Lys, Asp, or Glu or to unnatural amino acids that contain similar reactive sidechain moieties.

Various size PEG groups can be used, as exemplified but not limited to, PEG polymers of from about 5 kDa to about 43 kDa. The PEG modification may include a single, linear PEG. For example, linear 5, 20, or 30 kDa PEGs that are attached to maleidmide or other cross-linking groups are available from Nektar and/or NOF. Also, the modification may involve branched PEGs that contain two or more PEG polymer chains that are attached to maleimide or other cross-linking groups are available from Nektar and NOF.

The linker between the PEG and the peptide cross-linking group can be varied. For example, the commercially available thiol-reactive 40 kDa PEG (mPEG2-MAL) from Nektar (Huntsville, Ala.) employs a maleimide group for conjugation to Cys, and the maleimide group is attached to the PEG via a linker that contains a Lys. As a second example, the commercially available thiol-reactive 43 kDa PEG (GL2-400MA) from NOF employs a maleimide group for conjugation to Cys, and the maleimide group is attached to the PEG via a bi-substituted alkane linker. In addition, the PEG polymer can be attached directly to the maleimide, as exemplified by PEG reagents of molecular-weight 5 and 20 kDa available form Nektar Therapeutics (Huntsville, Ala.).

The polypeptides of the present invention include, for example, the polypeptides of FIG. 1 (SEQ ID NOs: 1 through 153), as well as those sequences having insubstantial variations in sequence from them. An “insubstantial variation” would include any sequence addition, substitution, or deletion variant that maintains substantially at least one biological function of the polypeptides of this invention, such as VPAC2 agonist activity and/or the insulin secreting activity demonstrated herein. These functional equivalents may include, for example, polypeptides which have at least about 90% identity to the polypeptides of FIG. 1, or at least 95% identity to the polypeptides of FIG. 1, or at least 97% identity to the polypeptides of FIG. 1, and also include portions of such polypeptides having substantially the same biological activity. However, any polypeptide having insubstantial variation in amino acid sequence from the polypeptides of FIG. 1 that demonstrates functional equivalency as described further herein is included in the description of the present invention.

As known in the art “similarity” between two polypeptides is determined by comparing the amino acid sequence and its conserved amino acid substitutes of one polypeptide to the sequence of a second polypeptide. Such conservative substitutions include those described above and by Dayhoff (The Atlas of Protein Sequence and Structure 5, 1978), and by Argos (EMBO J. 8:779-785, 1989). For example, amino acids belonging to one of the following groups represent conservative changes:
ala, pro, gly, gin, asn, ser, thr;
cys, ser, tyr, thr;
val, ile, leu, met, ala, phe;
lys, arg, his;
phe, tyr, trp, his; and
asp, glu.

Also provided are related compounds within the understanding of those with skill in the art, such as chemical mimetics, organomimetics, or peptidomimetics. As used herein, the terms “mimetic,” “peptide mimetic,” “peptidomimetic,” “organomimetic,” and “chemical mimetic” are intended to encompass peptide derivatives, peptide analogs, and chemical compounds having an arrangement of atoms in a three-dimensional orientation that is equivalent to that of a peptide of the present invention. It will be understood that the phrase “equivalent to” as used herein is intended to encompass compounds having substitution(s) of certain atoms, or chemical moieties in said peptide, having bond lengths, bond angles, and arrangements in the mimetic compound that produce the same or sufficiently similar arrangement or orientation of said atoms and moieties to have the biological function of the peptides of the invention. In the peptide mimetics of the invention, the three-dimensional arrangement of the chemical constituents is structurally and/or functionally equivalent to the three-dimensional arrangement of the peptide backbone and component amino acid sidechains in the peptide, resulting in such peptido-, organo-, and chemical mimetics of the peptides of the invention having substantial biological activity. These terms are used according to the understanding in the art, as illustrated, for example, by Fauchere, (Adv. Drug Res. 15:29,1986); Veber & Freidinger, (TINS p.392,1985); and Evans, et al., (J. Med. Chem. 30:1229, 1987), incorporated herein by reference.

It is understood that a pharmacophore exists for the biological activity of each peptide of the invention. A pharmacophore is understood in the art as comprising an idealized, three-dimensional definition of the structural requirements for biological activity. Peptido-, organo-, and chemical mimetics may be designed to fit each pharmacophore with current computer modeling software (computer aided drug design). Said mimetics may be produced by structure-function analysis, based on the positional information from the substituent atoms in the peptides of the invention.

Peptides as provided by the invention can be advantageously synthesized by any of the chemical synthesis techniques known in the art, particularly solid-phase synthesis techniques, for example, using commercially-available automated peptide synthesizers. The mimetics of the present invention can be synthesized by solid phase or solution phase methods conventionally used for the synthesis of peptides (see, e.g., Merrifield, J. Amer. Chem. Soc. 85:2149-54,1963; Carpino, Acc. Chem. Res. 6:191-98, 1973; Birr, Aspects of the Merrifield Peptide Synthesis, Springer-Verlag: Heidelberg, 1978; The Peptides: Analysis, Synthesis, Biology, Vols. 1, 2, 3, and 5, (Gross & Meinhofer, eds.), Academic Press: New York, 1979; Stewart, et al., Solid Phase Peptide Synthesis, 2nd. ed., Pierce Chem. Co.: Rockford, Ill., 1984; Kent, Ann. Rev. Biochem. 57:957-89,1988; and Gregg, et al., Int. J. Peptide Protein Res. 55:161-214, 1990, which are incorporated herein by reference in their entirety.)

The solid phase methodology may also be utilized. Briefly, an N-protected C-terminal amino acid residue is linked to an insoluble support such as divinylbenzene cross-linked polystyrene, polyacrylamide resin, Kieselguhr/polyamide (pepsyn K), controlled pore glass, cellulose, polypropylene membranes, acrylic acid-coated polyethylene rods, or the like. Cycles of deprotection, neutralization, and coupling of successive protected amino acid derivatives are used to link the amino acids from the C-terminus according to the amino acid sequence. For some synthetic peptides, an FMOC strategy using an acid-sensitive resin may be used. Examples of solid supports in this regard are divinylbenzene cross-linked polystyrene resins, which are commercially available in a variety of functionalized forms, including chloromethyl resin, hydroxymethyl resin, paraacetamidomethyl resin, benzhydrylamine (BHA) resin, 4-methylbenzhydrylamine (MBHA) resin, oxime resins, 4-alkoxybenzyl alcohol resin (Wang resin), 4-(2′,4′-dimethoxyphenylaminomethyl)-phenoxymethyl resin, 2,4-dimethoxybenzhydryl-amine resin, and 4-(2′,4′-dimethoxyphenyl-FMOC-amino-methyl)-phenoxyacetamidonorleucyl-MBHA resin (Rink amide MBHA resin). In addition, acid-sensitive resins also provide C-terminal acids, if desired. A particularly preferred protecting group for alpha amino acids is base-labile 9-fluorenylmethoxy-carbonyl (FMOC).

Suitable protecting groups for the side chain functionalities of amino acids chemically compatible with BOC (t-butyloxycarbonyl) and FMOC groups are well known in the art. When using FMOC chemistry, the following protected amino acid derivatives are preferred: FMOC-Cys(Trit), FMOC-Ser(But), FMOC-Asn(Trit), FMOC-Leu, FMOC-Thr(Trit), FMOC-Val, FMOC-Gly, FMOC-Lys(Boc), FMOC-Gln(Trit), FMOC-Glu(OBut), FMOC-His(Trit), FMOC-Tyr(But), FMOC-Arg(PMC (2,2,5,7,8-pentamethylchroman-6-sulfonyl)), FMOC-Arg(BOC)₂, FMOC-Pro, and FMOC-Trp(BOC). The amino acid residues may be coupled by using a variety of coupling agents and chemistries known in the art, such as direct coupling with DIC (diisopropyl-carbodiimide), DCC (dicyclohexylcarbodiimide), BOP (benzotriazolyl-N-oxytrisdimethylaminophosphonium hexa-fluorophosphate), PyBOP (benzotriazole-1-yl-oxy-tris-pyrrolidinophosphonium hexafluoro-phosphate), PyBrOP (bromo-tris-pyrrolidinophosphonium hexafluorophosphate); via performed symmetrical anhydrides; via active esters such as pentafluorophenyl esters; or via performed HOBt (1-hydroxybenzotriazole) active esters or by using FMOC-amino acid fluoride and chlorides or by using FMOC-amino acid-N-carboxy anhydrides. Activation with HBTU (2-(1 H-benzotriazole-1-yl),1,1,3,3-tetramethyluronium hexafluorophosphate) or HATU (2-(1H-7-aza-benzotriazole-1-yl), 1,1,3,3-tetramethyluronium hexafluoro-phosphate) in the presence of HOBt or HOAt (7-azahydroxybenztriazole) is preferred.

The solid phase method may be carried out manually, or automated synthesis on a commercially available peptide synthesizer (e.g., Applied Biosystems 431A or the like; Applied Biosystems, Foster City, Calif.) may be used. In a typical synthesis, the first (C-terminal) amino acid is loaded on the chlorotrityl resin. Successive deprotection (with 20% piperidine/NMP (N-methylpyrrolidone)) and coupling cycles according to ABI FastMoc protocols (Applied Biosystems) may be used to generate the peptide sequence. Double and triple coupling, with capping by acetic anhydride, may also be used.

The synthetic mimetic peptide may be cleaved from the resin and deprotected by treatment with TFA (trifluoroacetic acid) containing appropriate scavengers. Many such cleavage reagents, such as Reagent K (0.75 g crystalline phenol, 0.25 mL ethanedithiol, 0.5 mL thioanisole, 0.5 mL deionized water, 10 mL TFA) and others, may be used. The peptide is separated from the resin by filtration and isolated by ether precipitation. Further purification may be achieved by conventional methods, such as gel filtration and reverse phase HPLC (high performance liquid chromatography). Synthetic mimetics according to the present invention may be in the form of pharmaceutically acceptable salts, especially base-addition salts including salts of organic bases and inorganic bases. The base-addition salts of the acidic amino acid residues are prepared by treatment of the peptide with the appropriate base or inorganic base, according to procedures well known to those skilled in the art, or the desired salt may be obtained directly by lyophilization of the appropriate base.

Generally, those skilled in the art will recognize that peptides as described herein may be modified by a variety of chemical techniques to produce peptides having essentially the same activity as the unmodified peptide, and optionally having other desirable properties. For example, carboxylic acid groups of the peptide may be provided in the form of a salt of a pharmaceutically-acceptable cation. Amino groups within the peptide may be in the form of a pharmaceutically-acceptable acid addition salt, such as the HCl, HBr, acetic, benzoic, toluene sulfonic, maleic, tartaric, and other organic salts, or may be converted to an amide. Thiols may be protected with any one of a number of well-recognized protecting groups, such as acetamide groups. Those skilled in the art will also recognize methods for introducing cyclic structures into the peptides of this invention so that the native binding configuration will be more nearly approximated. For example, a carboxyl terminal or amino terminal cysteine residue may be added to the peptide, so that when oxidized the peptide will contain a disulfide bond, thereby generating a cyclic peptide. Other peptide cyclizing methods include the formation of thioethers and carboxyl- and amino-terminal amides and esters.

Specifically, a variety of techniques are available for constructing peptide derivatives and analogs with the same or similar desired biological activity as the corresponding peptide compound but with more favorable activity than the peptide with respect to solubility, stability, and susceptibility to hydrolysis and proteolysis. Such derivatives and analogs include peptides modified at the N-terminal amino group, the C-terminal carboxyl group, and/or changing one or more of the amido linkages in the peptide to a non-amido linkage. It will be understood that two or more such modifications may be coupled in one peptide mimetic structure (e.g., modification at the C-terminal carboxyl group and inclusion of a —CH₂- carbamate linkage between two amino acids in the peptide).

Amino terminus modifications include alkylating, acetylating, adding a carbobenzoyl group, and forming a succinimide group. Specifically, the N-terminal amino group may be reacted to form an amide group of the formula RC(O)NH— where R is alkyl, preferably lower alkyl, and is added by reaction with an acid halide, RC(O)Cl or acid anhydride. Typically, the reaction can be conducted by contacting about equimolar or excess amounts (e.g., about 5 equivalents) of an acid halide to the peptide in an inert diluent (e.g., dichloromethane) preferably containing an excess (e.g., about 10 equivalents) of a tertiary amine, such as diisopropylethylamine, to scavenge the acid generated during reaction. Reaction conditions are otherwise conventional (e.g., room temperature for 30 minutes). Alkylation of the terminal amino to provide for a lower alkyl N-substitution followed by reaction with an acid halide as described above will provide an N-alkyl amide group of the formula RC(O)NR—. Alternatively, the amino terminus may be covalently linked to succinimide group by reaction with succinic anhydride. An approximately equimolar amount or an excess of succinic anhydride (e.g., about 5 equivalents) is used and the terminal amino group is converted to the succinimide by methods well known in the art including the use of an excess (e.g., 10 equivalents) of a tertiary amine such as diisopropylethylamine in a suitable inert solvent (e.g., dichloromethane), as described in Wollenberg, et al., (U.S. Pat. No. 4,612,132), and is incorporated herein by reference in its entirety. It will also be understood that the succinic group may be substituted with, for example, a C₂- through C₆- alkyl or —SR substituents, which are prepared in a conventional manner to provide for substituted succinimide at the N-terminus of the peptide. Such alkyl substituents may be prepared by reaction of a lower olefin (C₂- through C₆-alkyl) with maleic anhydride in the manner described by Wollenberg, et al., supra., and —SR substituents may be prepared by reaction of RSH with maleic anhydride where R is as defined above. In another advantageous embodiment, the amino terminus may be derivatized to form a benzyloxycarbonyl-NH— or a substituted benzyloxycarbonyl-NH— group. This derivative may be produced by reaction with approximately an equivalent amount or an excess of benzyloxycarbonyl chloride (CBZ-Cl), or a substituted CBZ-Cl in a suitable inert diluent (e.g., dichloromethane) preferably containing a tertiary amine to scavenge the acid generated during the reaction. In yet another derivative, the N-terminus comprises a sulfonamide group by reaction with an equivalent amount or an excess (e.g., 5 equivalents) of R—S(O)₂Cl in a suitable inert diluent (dichloromethane) to convert the terminal amine into a sulfonamide, where R is alkyl and preferably lower alkyl. Preferably, the inert diluent contains excess tertiary amine (e.g., 10 equivalents) such as diisopropylethylamine, to scavenge the acid generated during reaction. Reaction conditions are otherwise conventional (e.g., room temperature for 30 minutes). Carbamate groups may be produced at the amino terminus by reaction with an equivalent amount or an excess (e.g., 5 equivalents) of R—OC(O)Cl or R—OC(O)OC₆H₄—p—NO₂ in a suitable inert diluent (e.g., dichloromethane) to convert the terminal amine into a carbamate, where R is alkyl, preferably lower alkyl. Preferably, the inert diluent contains an excess (e.g., about 10 equivalents) of a tertiary amine, such as diisopropylethylamine, to scavenge any acid generated during reaction. Reaction conditions are otherwise conventional (e.g., room temperature for 30 minutes). Urea groups may be formed at the amino terminus by reaction with an equivalent amount or an excess (e.g., 5 equivalents) of R—N═C═O in a suitable inert diluent (e.g., dichloromethane) to convert the terminal amine into a urea (i.e., RNHC(O)NH—) group where R is as defined above. Preferably, the inert diluent contains an excess (e.g., about 10 equivalents) of a tertiary amine, such as diisopropylethylamine. Reaction conditions are otherwise conventional (e.g., room temperature for about 30 minutes).

In preparing peptide mimetics wherein the C-terminal carboxyl group may be replaced by an ester (e.g., —C(O)OR where R is alkyl and preferably lower alkyl), resins used to prepare the peptide acids may be employed, and the side chain protected peptide may be cleaved with a base and the appropriate alcohol (e.g., methanol). Side chain protecting groups may be removed in the usual fashion by treatment with hydrogen fluoride to obtain the desired ester. In preparing peptide mimetics wherein the C-terminal carboxyl group is replaced by the amide —C(O)NR₃R₄, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH₂). Alternatively, use of the chloromethylated resin during peptide synthesis coupled with reaction with ammonia to cleave the side chain protected peptide from the support yields the free peptide amide, and reaction with an alkylamine or a dialkylamine yields a side chain protected alkylamide or dialkylamide (i.e., the C-terminus is —C(O)NRR,, where R and R₁ are alkyl and preferably lower alkyl). Side chain protection is then removed in the usual fashion by treatment with hydrogen fluoride to give the free amides, alkylamides, or dialkylamides.

In another alternative embodiment, the C-terminal carboxyl group or a C-terminal ester may be induced to cyclize by displacement of the —OH or the ester (—OR) of the carboxyl group or ester, respectively, with the N-terminal amino group to form a cyclic peptide. For example, after synthesis and cleavage to give the peptide acid, the free acid is converted in solution to an activated ester by an appropriate carboxyl group activator such as dicyclohexylcarbodiimide (DCC), for example, in methylene chloride (CH₂Cl₂), dimethyl formamide (DMF), or mixtures thereof. The cyclic peptide is then formed by displacement of the activated ester with the N-terminal amine. Cyclization, rather than polymerization, may be enhanced by use of very dilute solutions according to methods well known in the art.

Peptide mimetics as understood in the art and provided by the invention are structurally similar to the peptide of the invention, but have one or more peptide linkages optionally replaced by a linkage selected from the group consisting of: —CH₂NH—, —CH₂S—, —CH₂CH₂—, —CH═CH— (in both cis and trans conformers), —COCH₂—, —CH(OH)CH₂—, and —CH₂SO—, by methods known in the art and further described in the following references: Spatola, Chemistry and Biochemistry of Amino Acids, Peptides, and Proteins, (Weinstein, ed.), Marcel Dekker: New York, p. 267, 1983; Spatola, Peptide Backbone Modifications 1:3,1983; Morley, Trends Pharm. Sci. pp. 463-468, 1980; Hudson, et al., Int. J. Pept. Prot. Res. 14:177-185, 1979; Spatola, et al., Life Sci. 38:1243-1249, 1986; Hann, J. Chem. Soc. Perkin Trans. I 307-314,1982; Almquist, et al., J. Med. Chem. 23:1392-1398,1980; Jennings-White, et al., Tetrahedron Lett. 23:2533,1982; Szelke, et al., EP045665A; Holladay, et al., Tetrahedron Left. 24:4401-4404, 1983; and Hruby, Life Sci. 31:189-199, 1982; each of which is incorporated herein by reference. Such peptide mimetics may have significant advantages over polypeptide embodiments, including, for example, more economical to produce, having greater chemical stability or enhanced pharmacological properties (such as half-life, absorption, potency, efficacy, etc.), reduced antigenicity, and other properties.

Mimetic analogs of the peptides of the invention may also be obtained using the principles of conventional or rational drug design (see, e.g., Andrews, et al., Proc. Alfred Benzon Symp. 28:145-165, 1990; McPherson, Eur. J. Biochem. 189:1-24,1990; Hol, et al., in Molecular Recognition: Chemical and Biochemical Problems, (Roberts, ed.); Royal Society of Chemistry; pp. 84-93, 1989a; Hol, Arzneim-Forsch. 39:1016-1018, 1989b; Hol, Agnew Chem. Int. Ed. Engl. 25:767-778, 1986; the disclosures of which are herein incorporated by reference).

In accordance with the methods of conventional drug design, the desired mimetic molecules may be obtained by randomly testing molecules whose structures have an attribute in common with the structure of a “native” peptide. The quantitative contribution that results from a change in a particular group of a binding molecule may be determined by measuring the biological activity of the putative mimetic in comparison with the activity of the peptide. In one embodiment of rational drug design, the mimetic is designed to share an attribute of the most stable three-dimensional conformation of the peptide. Thus, for example, the mimetic may be designed to possess chemical groups that are oriented in a way sufficient to cause ionic, hydrophobic, or van der Waals interactions that are similar to those exhibited by the peptides of the invention, as disclosed herein.

One method for performing rational mimetic design employs a computer system capable of forming a representation of the three-dimensional structure of the peptide, such as those exemplified by Hol, 1989a; Hol, 1989b; and Hol, 1986. Molecular structures of the peptido-, organo-, and chemical mimetics of the peptides of the invention may be produced using computer-assisted design programs commercially available in the art. Examples of such programs include SYBYL 6.5®, HQSAR™, and ALCHEMY 2000™ (Tripos); GALAXY™ and AM2000™ (AM Technologies, Inc., San Antonio, Tex.); CATALYST™ and CERIUS™ (Molecular Simulations, Inc., San Diego, Calif.); CACHE PRODUCTS™, TSAR™, AMBER™, and CHEM-X™ (Oxford Molecular Products, Oxford, Calif.) and CHEMBUILDER3D™ (Interactive Simulations, Inc., San Diego, Calif.).

The peptido-, organo-, and chemical mimetics produced using the peptides disclosed herein using, for example, art-recognized molecular modeling programs may be produced using conventional chemical synthetic techniques, methods designed to accommodate high throughput screening, including combinatorial chemistry methods. Combinatorial methods useful in the production of the peptido-, organo-, and chemical mimetics of the invention include phage display arrays, solid-phase synthesis, and combinatorial chemistry arrays, as provided, for example, by SIDDCO (Tuscon, Ariz.); Tripos, Inc.; Calbiochem/Novabiochem (San Diego, Calif.); Symyx Technologies, Inc. (Santa Clara, Calif.); Medichem Research, Inc. (Lemont, Ill.); Pharm-Eco Laboratories, Inc. (Bethlehem, Pa.); or N. V. Organon (Oss, Netherlands). Combinatorial chemistry production of the peptido-, organo-, and chemical mimetics of the invention may be produced according to methods known in the art, including, but not limited to, techniques disclosed in Terreft, (Combinatorial Chemistry, Oxford University Press, London, 1998); Gallop, et al., J. Med. Chem. 37:1233-51, 1994; Gordon, et al., J. Med. Chem. 37:1385-1401, 1994; Look, et al., Bioorg. Med. Chem. Lett. 6:707-12, 1996; Ruhland, et al., J. Amer. Chem. Soc. 118: 253-4,1996; Gordon, et al., Acc. Chem. Res. 29:144-54,1996; Thompson & Ellman, Chem. Rev. 96:555-600,1996; Fruchtel & Jung, Angew. Chem. Int. Ed. Engl. 35:17-42,1996; Pavia, “The Chemical Generation of Molecular Diversity”, Network Science Center, www.netsci.org, 1995; Adnan, et al., “Solid Support Combinatorial Chemistry in Lead Discovery and SAR Optimization,” Id., 1995; Davies and Briant, “Combinatorial Chemistry Library Design using Pharmacophore Diversity,” Id., 1995; Pavia, “Chemically Generated Screening Libraries: Present and Future,” Id., 1996; and U.S. Pat. Nos. 5,880,972; 5,463,564; 5,331573; and 5,573,905.

The newly synthesized polypeptides may be substantially purified by preparative high performance liquid chromatography (see, e.g., Creighton, Proteins: Structures And Molecular Principles, W H Freeman and Co., New York, N.Y., 1983). The composition of a synthetic polypeptide of the present invention may be confirmed by amino acid analysis or sequencing by, for example, the Edman degradation procedure (Creighton, supra). Additionally, any portion of the amino acid sequence of the polypeptide may be altered during direct synthesis and/or combined using chemical methods with sequences from other proteins to produce a variant polypeptide or a fusion polypeptide.

Also included in this invention are antibodies and antibody fragments that selectively bind the polypeptides of this invention. Any type of antibody known in the art may be generated using methods well known in the art. For example, an antibody may be generated to bind specifically to an epitope of a polypeptide of this invention. “Antibody” as used herein includes intact immunoglobulin molecules, as well as fragments thereof, such as Fab, F(ab′)₂, and Fv, which are capable of binding an epitope of a polypeptide of this invention. Typically, at least 6, 8, 10, or 12 contiguous amino acids are required to form an epitope. However, epitopes which involve non-contiguous amino acids may require more amino acids, for example, at least 15, 25, or 50 amino acids.

An antibody which specifically binds to an epitope of a polypeptide of this invention may be used therapeutically, as well as in immunochemical assays, such as Western blots, ELISAs, radioimmunoassays, immunohistochemical assays, immunoprecipitations, or other immunochemical assays known in the art. Various immunoassays may be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a polypeptide of this invention provides a detection signal at least 5-, 10-, or 20-fold higher than a detection signal provided with other proteins when used in an immunochemical assay. For example, antibodies which specifically bind to a polypeptide of this invention do not detect other proteins in immunochemical assays and can immunoprecipitate a polypeptide of this invention from solution.

Polypeptides of this invention may be used to immunize a mammal, such as a mouse, rat, rabbit, guinea pig, monkey, or human, to produce polyclonal antibodies. If desired, a polypeptide of this invention may be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g., lysolecithin, pluronic polyols, polyanions, peptides, oil emulsions, keyhole limpet hemocyanin, and dinitrophenol). Among adjuvants used in humans, BCG (bacilli Calmeffe-Guerin) and Corynebacterium parvum are especially useful.

Monoclonal antibodies which specifically bind to a polypeptide of this invention may be prepared using any technique which provides for the production of antibody molecules by continuous cell lines in culture. These techniques include, but are not limited to, the hybridoma technique, the human B cell hybridoma technique, and the EBV hybridoma technique (Kohler, et al., Nature 256:495-97,1985; Kozbor, et al., J. Immunol. Methods 81:3142,1985; Cote, et al., Proc. Natl. Acad. Sci. 80:2026-30, 1983; Cole, et al., Mol. Cell Biol. 62:109-20,1984).

In addition, techniques developed for the production of “chimeric antibodies,” the splicing of mouse antibody genes to human antibody genes to obtain a molecule with appropriate antigen specificity and biological activity, may be used (Morrison, et al., Proc. Natl. Acad. Sci. 81:6851-55, 1984; Neuberger, et al., Nature 312:604-08,1984; Takeda, et al., Nature 314:452-54,1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences may be minimized by replacing residues which differ from those in the human sequences by site directed mutagenesis of individual residues or by grating of entire complementarity determining regions. Alternatively, humanized antibodies may be produced using recombinant methods (see, e.g., GB2188638B). Antibodies which specifically bind to a polypeptide of this invention may contain antigen binding sites which are either partially or fully humanized, as disclosed in U.S. Pat. No. 5,565,332.

Alternatively, techniques described for the production of single chain antibodies may be adapted using methods known in the art to produce single chain antibodies which specifically bind to a polypeptide of this invention. Antibodies with related specificity, but of distinct idiotypic composition, can be generated by chain shuffling from random combinatorial immunoglobin libraries (Burton, Proc. Natl. Acad. Sci. 88:11120-23,1991).

Single-chain antibodies also may be constructed using a DNA amplification method, such as PCR, using hybridoma cDNA as a template (Thirion, et al., Eur. J. Cancer Prev. 5:507-11, 1996). Single-chain antibodies can be mono- or bispecific, and can be bivalent or tetravalent. Construction of tetravalent, bispecific single-chain antibodies is taught, for example, in Coloma & Morrison (Nat. Biotechnol. 15:159-63,1997). Construction of bivalent, bispecific single-chain antibodies is taught in Mallender & Voss (J. Biol. Chem. 269:199-206,1994).

A nucleotide sequence encoding a single-chain antibody may be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar, et al., Int. J. Cancer 61:497-501,1995; Nicholls, et al., J. Immunol. Meth. 165:81-91, 1993).

Antibodies which specifically bind to a polypeptide of this invention may also be produced by inducing in vivo production in the lymphocyte population or by screening immunoglobulin libraries or panels of highly specific binding reagents as disclosed in the literature (Orlandi, et al., Proc. Natl. Acad. Sci. 86:38333-37, 1989; Winter, et al., Nature 349:293-99,1991).

Other types of antibodies may be constructed and used therapeutically in methods of the invention. For example, chimeric antibodies may be constructed as disclosed in WO 93/03151. Binding proteins which are derived from immunoglobulins and which are multivalent and multispecific, such as the “diabodies” also can be prepared (see, e.g., WO 94/13804,).

Human antibodies with the ability to bind to the polypeptides of this invention may also be identified from the MorphoSys HuCAL® library, or similar technology, as follows. A polypeptide of this invention may be coated on a microtiter plate and incubated with the MorphoSys HuCAL® Fab phage library. Those phage-linked Fabs not binding to the polypeptide of this invention can be washed away from the plate, leaving only phage which tightly bind to the polypeptide of this invention. The bound phage can be eluted, for example, by a change in pH or by elution with E. coli and amplified by infection of E. coli hosts. This panning process can be repeated once or twice to enrich for a population of antibodies that tightly bind to the polypeptide of this invention. The Fabs from the enriched pool are then expressed, purified, and screened in an ELISA assay.

Antibodies according to the invention may be purified by methods well known in the art. For example, antibodies may be affinity purified by passage over a column to which a polypeptide of this invention is bound. The bound antibodies can then be eluted from the column using a buffer with a high salt concentration.

### Methods of Use

As used herein, various terms are defined below.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “subject” as used herein includes mammals (e.g., humans and animals).

The term “treatment” includes any process, action, application, therapy, or the like, wherein a subject, including a human being, is provided medical aid with the object of improving the subject's condition, directly or indirectly, or slowing the progression of a condition or disorder in the subject.

The term “combination therapy” or “co-therapy” means the administration of two or more therapeutic agents to treat a diabetic condition and/or disorder. Such administration encompasses co-administration of two or more therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner.

The phrase “therapeutically effective” means the amount of each agent administered that will achieve the goal of improvement in a diabetic condition or disorder severity, while avoiding or minimizing adverse side effects associated with the given therapeutic treatment.

The term “pharmaceutically acceptable” means that the subject item is appropriate for use in a pharmaceutical product.

The polypeptides of the present invention are expected to be valuable as therapeutic agents. Accordingly, an embodiment of this invention includes a method of treating the various conditions in a patient (including mammals) which comprises administering to said patient a composition containing an amount of a polypeptide of the present invention, that is effective in treating the target condition.

The polypeptides of the present invention, as a result of the ability to stimulate insulin secretion from pancreatic islet cells in vitro, and by causing a decrease in blood glucose in vivo, may be employed in treatment diabetes, including both type 1 and type 2 diabetes (non-insulin dependent diabetes mellitus). Such treatment may also delay the onset of diabetes and diabetic complications. The polypeptides may be used to prevent subjects with impaired glucose tolerance from proceeding to develop type 2 diabetes. Other diseases and conditions that may be treated or prevented using polypeptides of the invention in methods of the invention include: Maturity-Onset Diabetes of the Young (MODY) (Herman, et al., Diabetes 43:40,1994); Latent Autoimmune Diabetes Adult (LADA) (Zimmet, et al., Diabetes Med. 11 :299, 1994); impaired glucose tolerance (IGT) (Expert Committee on Classification of Diabetes Mellitus, Diabetes Care 22 (Supp. 1):S5, 1999); impaired fasting glucose (IFG) (Charles, et al., Diabetes 40:796, 1991); gestational diabetes (Metzger, Diabetes, 40:197,1991); and metabolic syndrome X.

The polypeptides of the present invention may also be utilized in the prevention and/or treatment of obesity (e.g., regulation of appetite and food intake); disorders of energy homeostasis; disorders of lipid and carbohydrate metabolism; cardiovascular disease, including atherosclerosis, coronary heart disease, coronary artery disease, hyperlipidemia, hypercholesteremia, low HDL levels and hypertension; cerebrovascular disease and peripheral vessel disease; polycystic ovary syndrome; carcinogenesis, and hyperplasia; asthma and chronic obstructive pulmonary disease; male reproduction problems (including erectile dysfunction); ulcers; neurodegenerative diseases (including Parkinson's and Alzheimer's); sleep disorders and circadian dysfunction; growth disorders; immune diseases, including autoimmune diseases (e.g., systemic lupus erythematosus); chronic inflammatory diseases; septic shock; HIV infection and AIDS, and other conditions identified herein, or function otherwise as described later herein.

The polypeptides of the present invention may also be useful for treating physiological disorders related to, for example, cell differentiation to produce lipid accumulating cells, regulation of insulin sensitivity and blood glucose levels, which are involved in, for example, abnormal pancreatic β-cell function, macrophage differentiation which leads to the formation of atherosclerotic plaques, inflammatory response, carcinogenesis, hyperplasia, reduction in the pancreatic β-cell mass, insulin secretion, tissue sensitivity to insulin, liposarcoma cell growth, polycystic ovarian disease, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL, and LDL cholesterol levels, and the like.

The polypeptides of the invention may also be used in methods of the invention to treat secondary causes of diabetes (Expert Committee on Classification of Diabetes Mellitus, Diabetes Care 22 (Supp. 1):S5, 1999). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenytoin, thyroid hormone, β-adrenergic agents, α-interferon and drugs used to treat HIV infection.

In addition, the polypeptides of the invention may be used for treatment of asthma (Bolin, et al., Biopolymer 37:57-66,1995; U.S. Pat. No. 5,677,419; showing that polypeptide R3P0 is active in relaxing guinea pig tracheal smooth muscle); hypotension induction (VIP induces hypotension, tachycardia, and facial flushing in asthmatic patients (Morice, et al., Peptides 7:279-280, 1986; Morice, et al., Lancet 2:1225-1227,1983); male reproduction problems (Siow, et al., Arch. Androl. 43(1):67-71, 1999); as an anti-apoptosis/neuroprotective agent (Brenneman, et al., Ann. N. Y. Acad. Sci. 865:207-12,1998); cardioprotection during ischemic events (Kalfin, et al., J. Pharmacol. Exp. Ther. 1268(2):952-8, 1994; Das, et al., Ann. N. Y. Acad. Sci. 865:297-308,1998), manipulation of the circadian clock and its associated disorders (Hamar, et al.,. Cell 109:497-508, 2002; Shen, et al., Proc. Natl. Acad. Sci. 97:11575-80, 2000), and finally as an anti-ulcer agent (Tuncel, et al., Ann. N. Y. Acad. Sci. 865:309-22,1998).

The polypeptides of the present invention may be used alone or in combination with additional therapies and/or compounds known to those skilled in the art in the treatment of diabetes and related disorders. Alternatively, the methods and polypeptides described herein may be used, partially or completely, in combination therapy.

The polypeptides of the invention may also be administered in combination with other known therapies for the treatment of diabetes, including PPAR ligands (e.g., agonists, antagonists), insulin secretagogues, for example, sulfonylurea drugs and non-sulfonylurea secretagogues, α-glucosidase inhibitors, insulin sensitizers, insulin secretagogues, hepatic glucose output lowering compounds, insulin and insulin derivatives, and anti-obesity drugs. Such therapies may be administered prior to, concurrently with, or following administration of the polypeptides of the invention. Insulin and insulin derivatives include both long and short acting forms and formulations of insulin. PPAR ligands may include agonists and/or antagonists of any of the PPAR receptors or combinations thereof. For example, PPAR ligands may include ligands of PPAR-α, PPAR-γ, PPAR-δ or any combination of two or three of the receptors of PPAR. PPAR ligands include, for example, rosiglitazone, troglitazone, and pioglitazone. Sulfonylurea drugs include, for example, glyburide, glimepiride, chlorpropamide, tolbutamide, and glipizide. α-glucosidase inhibitors that may be useful in treating diabetes when administered with a polypeptide of the invention include acarbose, miglitol, and voglibose. Insulin sensitizers that may be useful in treating diabetes include PPAR-γ agonists such as the glitazones (e.g., troglitazone, pioglitazone, englitazone, MCC-555, rosiglitazone, and the like) and other thiazolidinedione and non-thiazolidinedione compounds; biguanides such as metformin and phenformin; protein tyrosine phosphatase-1B (PTP-1B) inhibitors; dipeptidyl peptidase IV (DPPIV) inhibitors; and 11beta-HSD inhibitors. Hepatic glucose output lowering compounds that may be useful in treating diabetes when administered with a peptide of the invention include, for example, glucagon anatgonists and metformin, such as Glucophage and Glucophage XR. Insulin secretagogues that may be useful in treating diabetes when administered with a peptide of the invention include sulfonylurea and non-sulfonylurea drugs: GLP-1, GIP, VIP, PACAP, secretin, and derivatives thereof; nateglinide, meglitinide, repaglinide, glibenclamide, glimepiride, chlorpropamide, and glipizide. For example, GLP-1 includes derivatives of GLP-1 with longer half-lives than native GLP-1, such as, for example, fatty-acid derivatized GLP-1 and exendin. In one embodiment of the invention, polypeptides of the invention are used in combination with insulin secretagogues to increase the sensitivity of pancreatic β-cells to the insulin secretagogue.

Polypeptides of the invention may also be used in methods of the invention in combination with anti-obesity drugs. Anti-obesity drugs include β-3 adrenergic receptor agonists; CB-1 (cannabinoid) receptor antagonists; neuropeptide Y antagonists; appetite suppressants, such as, for example, sibutramine (Meridia); and lipase inhibitors, such as, for example, orlistat (Xenical). The polypeptides of the present invention may be administered in combination with other pharmaceutical agents, such as apo-B/MTP inhibitors, MCR-4 agonists, CCK-A agonists, monoamine reuptake inhibitors, sympathomimetic agents, dopamine agonists, melanocyte-stimulating hormone receptor analogs, melanin concentrating hormone antagonists, leptins, leptin analogs, leptin receptor agonists, galanin antagonists, lipase inhibitors, bombesin agonists, thyromimetic agents, dehydroepiandrosterone or analogs thereof, glucocorticoid receptor agonists or antagonists, orexin receptor antagonists, urocortin binding protein antagonists, ciliary neurotrophic factors, AGRPs (human agouti-related proteins), ghrelin receptor antagonists, histamine 3 receptor antagonists or reverse agonists, neuromedin U receptor agonists, and the like.

Polypeptides of the invention may also be used in methods of the invention in combination with drugs commonly used to treat lipid disorders. Such drugs include, but are not limited to, HMG-CoA reductase inhibitors, nicotinic acid, fatty acid lowering compounds (e.g., acipimox); lipid lowering drugs (e.g., stanol esters, sterol glycosides such as tiqueside, and azetidinones such as ezetimibe), ACAT inhibitors (such as avasimibe), bile acid sequestrants, bile acid reuptake inhibitors, microsomal triglyceride transport inhibitors, and fibric acid derivatives. HMG-CoA reductase inhibitors include, for example, lovastatin, simvastatin, pravastatin, fluvastatin, atorvastatin, rivastatin, itavastatin, cerivastatin, and ZD-4522. Fibric acid derivatives include, for example, clofibrate, fenofibrate, bezafibrate, ciprofibrate, beclofibrate, etofibrate, and gemfibrozil. Sequestrants include, for example, cholestyramine, colestipol, and dialkylaminoalkyl derivatives of a cross-linked dextran.

Furthermore, polypeptides of the invention may also be administered in combination with anti-hypertensive drugs, such as, for example, β-blockers and ACE inhibitors. Examples of additional anti-hypertensive agents for use in combination with the peptides of the present invention include calcium channel blockers (L-type and T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e. g., losartan, irbesartan, valsartan), ET receptor antagonists (e.g., sitaxsentan, atrsentan, neutral endopeptidase (NEP) inhibitors, vasopepsidase inhibitors (dual NEP-ACE inhibitors) (e.g., omapatrilat and gemopatrilat), and nitrates.

Such co-therapies may be administered in any combination of two or more drugs (e.g., a polypeptide of the invention in combination with an insulin sensitizer and an anti-obesity drug). Such co-therapies may be administered in the form of pharmaceutical compositions, as described above.

### Pharmaceutical Compositions

Based on well known assays used to determine the efficacy for treatment of conditions identified above in mammals, and by comparison of these results with the results of known medicaments that are used to treat these conditions, the effective dosage of the polypeptides of this invention can readily be determined for treatment of each desired indication. The amount of the active ingredient (e.g., polypeptides) to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.0001 mg/kg to about 200 mg/kg, and preferably from about 0.01 mg/kg to about 200 mg/kg body weight per day. A unit dosage may contain from about 0.05 mg to about 1500 mg of active ingredient, and may be administered one or more times per day. The daily dosage for administration by injection, including intravenous, intramuscular, subcutaneous, and parenteral injections, and use of infusion techniques may be from about 0.01 to about 200 mg/kg. The daily rectal dosage regimen may be from 0.01 to 200 mg/kg of total body weight. The transdermal concentration may be that required to maintain a daily dose of from 0.01 to 200 mg/kg.

Of course, the specific initial and continuing dosage regimen for each patient will vary according to the nature and severity of the condition as determined by the attending diagnostician, the activity of the specific polypeptide employed, the age of the patient, the diet of the patient, time of administration, route of administration, rate of excretion of the drug, drug combinations, and the like. The desired mode of treatment and number of doses of a polypeptide of the present invention may be ascertained by those skilled in the art using conventional treatment tests.

The polypeptides of this invention may be utilized to achieve the desired pharmacological effect by administration to a patient in need thereof in an appropriately formulated pharmaceutical composition. A patient, for the purpose of this invention, is a mammal, including a human, in need of treatment for a particular condition or disease. Therefore, the present invention includes pharmaceutical compositions which are comprised of a pharmaceutically acceptable carrier and a therapeutically effective amount of a polypeptide. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A therapeutically effective amount of a polypeptide is that amount which produces a result or exerts an influence on the particular condition being treated. The polypeptides described herein may be administered with a pharmaceutically-acceptable carrier using any effective conventional dosage unit forms, including, for example, immediate and timed release preparations, orally, parenterally, topically, or the like.

For oral administration, the polypeptides may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the polypeptides of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or npropyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The polypeptides of this invention may also be administered parenterally, that is, subcutaneously, intravenously, intramuscularly, or interperitoneally, as injectable dosages of the polypeptide in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug (e.g., polypeptide) with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the polypeptides of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

Another formulation employs the use of biodegradable microspheres that allow controlled, sustained release of the peptides and PEGylated peptides of this invention. Such formulations can be comprised of synthetic polymers or copolymers. Such formulations allow for injection, inhalation, nasal or oral administration. The construction and use of biodegradable microspheres for the delivery of pharmaceutical agents is well known in the art (e.g., U.S. Pat. No. 6, 706,289, incorporated herein by reference).

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The polypeptides described herein may be administered as the sole pharmaceutical agent or in combination with one or more other pharmaceutical agents where the combination causes no unacceptable adverse effects. For example, the polypeptides of this invention can be combined with known anti-obesity, or with known antidiabetic or other indication agents, and the like, as well as with admixtures and combinations thereof.

The polypeptides described herein may also be utilized, in free base form or in compositions, in research and diagnostics, or as analytical reference standards, and the like. Therefore, the present invention includes compositions which are comprised of an inert carrier and an effective amount of a polypeptide identified by the methods described herein, or a salt or ester thereof. An inert carrier is any material which does not interact with the polypeptide to be carried and which lends support, means of conveyance, bulk, traceable material, and the like to the polypeptide to be carried. An effective amount of polypeptide is that amount which produces a result or exerts an influence on the particular procedure being performed.

Polypeptides are known to undergo hydrolysis, deamidation, oxidation, racemization and isomerization in aqueous and non-aqueous environment. Degradation such as hydrolysis, deamidation or oxidation can readily detected by capillary electrophoresis. Enzymatic degradation notwithstanding, polypeptides having a prolonged plasma half-life, or biological resident time, should, at minimum, be stable in aqueous solution. It is essential that polypeptide exhibits less than 10% degradation over a period of one day at body temperature. It is still more preferable that the polypeptide exhibits less than 5% degradation over a period of one day at body temperature. Because of the life time treatment in chronic diabetic patient, much preferably these therapeutic agents are convenient to administer, furthermore infrequently if by parenteral route. Stability (i.e., less than a few percent of degradation) over a period of weeks at body temperature will allow less frequent dosing. Stability in the magnitude of years at refrigeration temperature will allow the manufacturer to present a liquid formulation, thus avoid the inconvenience of reconstitution. Additionally, stability in organic solvent would provide polypeptide be formulated into novel dosage forms such as implant.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20th edition, 2000).

The structures, materials, compositions, and methods described herein are intended to be representative examples of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

The following examples are presented to illustrate the invention described herein, but should not be construed as limiting the scope of the invention in any way.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1

### Peptide Synthesis Methodology

The following general procedure was followed to synthesize some of the polypeptides of the invention:

Peptide synthesis was carried out by the FMOC/t-Butyl strategy (Pennington & Dunn, Peptide Synthesis Protocols, Volume 35, 1994) under continuous flow conditions using Rapp-Polymere PEG-Polystyrene resins (Rapp-Polymere, Tubingen, Germany). At the completion of synthesis, peptides are cleaved from the resin and de-protected using TFA/DTT/H₂0/Triisopropyl silane (88/5/5/2). Peptides were precipitated from the cleavage cocktail using cold diethyl ether. The precipitate was washed three times with the cold ether, and then dissolved in 5% acetic acid prior to lyophilization. Peptides were checked by reversed phase chromatography on a YMC-Pack ODS-AQ column (YMC, Inc., Wilmington, N.C.) on a Waters ALLIANCE® system (Waters Corporation, Milford, Mass.) using water/acetonitrile with 3% TFA as a gradient from 0% to 100% acetonitrile, and by MALDI mass spectrometry on a VOYAGER DE™ MALDI Mass Spectrometer, (Model 5-2386-00, PerSeptive BioSystems, Framingham, Mass.). The peptide sample was added to the Matrix buffer (50/50 dH₂O/acetonitrile with 3% TFA) in a 1/1 ratio. Those peptides not meeting the purity criteria of >95% are purified by reversed phase chromatography on a Waters Delta Prep 4000 HPLC System (Waters Corporation, Milford, Mass.).

### Example 2

### Peptide PEGylation

The half-life of a peptide in vivo may be increased through attachment of a polyethylene glycol (PEG) moiety to the peptide thereby reducing clearance of the peptide by the kidney and decreasing protease degradation of the peptide. The use of a VPAC2 receptor agonist peptide is severely limited by its very short half-life in vivo; however, attachment of a PEG moiety to the peptide (PEGylation) prolonged the half-life of the peptide sufficiently to allow for once/day to once/week treatment.

PEGylation may be performed by any method known to those skilled in the art. However, in this example, PEGylation was performed by introducing a unique cysteine mutation into the peptide followed by PEGylating the cysteine via a stable thioether linkage between the sulfhydryl of the peptide and maleimide group of the methoxy-PEG-maleimide reagent Nektar Therapeutics (Huntsville, Ala., USA). It is preferable to introduce the unique cysteine at the C-terminus of the peptide to minimize potential reduction of activity by PEGylation.

Specifically, a 2-fold molar excess of mPEG-mal (MW 22 kD and 43 kD) reagent was added to 1 mg of peptide (e.g., SEQ ID NO:1 having a cysteine mutation at the C-terminus of the peptide) and dissolved in reaction buffer at pH 6 (0.1M Na phosphate/0.1 M NaCl/0.1M EDTA). After 0.5 hour at room temperature, the reaction was terminated with 2-fold molar excess of DTT to mPEG-mal. The peptide-PEG-mal reaction mixture was applied to a cation exchange column to remove residual PEG reagents followed by gel filtration column to remove residual free peptide. The purity, mass, and number of PEGylated sites were determined by SDS-PAGE and MALDI-TOF mass spectrometry. When a 22 kD PEG was attached to peptides of the present invention, potent VPAC2 receptor activation was retained. Furthermore, VPAC2 versus VPAC1 and PAC1 selectivity of receptor activation was also retained. It is possible that PEGylation with a smaller PEG (e.g., a linear 22 kD PEG) will less likely reduce activity of the peptide, whereas a larger PEG (e.g., a branched 43 kD PEG) will more likely reduce activity. However, the larger PEG will increase plasma half-life further so that once a week injection may be possible (Harris, et al., Clin. Pharmacokinet. 40:539-551, 2001).

### Example 3

### Peptide Cloning

To express these peptides recombinantly, the DNA sequence encoding a peptide was cloned C-terminal to glutathione S-transferase (GST) with a single Factor Xa recognition site separating the monomeric peptide and GST. The gene encoding the Factor Xa recognition site fused to DNA sequence of the peptide to be produced was synthesized by hybridizing two overlapping single-stranded DNA fragments (70-90mers) containing a Bam HI or Xho I restriction enzyme site immediately 5′ to the DNA sequence of the gene to be cloned, followed by DNA synthesis of the opposite strands via the large fragment of DNA polymerase I (Life Technologies, Inc., Gaithersburg, Md.). The DNA sequence chosen for each gene was based on the reverse translation of the designed amino acid sequence of each peptide. In some cases, the gene encoding the peptide was generated by PCR mutagenesis (Picard, et al., Nucleic Acids Res 22:2587-91, 1994; Sambrook, et al., Molecular Cloning: A Laboratory Manual, 2nd ed., Cold Spring Harbor Laboratory Press, New York, 1989) of a gene already made by the method described above. The double-stranded product was then digested by Bam HI and Xho I and ligated into pGEX-6P-1 (Amersham Pharmacia Biotech, Piscataway, N.J.) which was also digested with Bam HI and Xho I.

### Example 4

### Peptide Recombinant Expression and Purification

BL21 cells (Stratagene, La Jolla, Calif.), transformed with the GST-peptide fusion-containing plasmids, were grown at 37° C. until the OD₆₀₀ reached 0.6 to 1.0, and then the cells were incubated with 1 mM IPTG (Life Technologies, Carlsbad, Calif.) for 2 hours at 37° C. Cells (2 L) were centrifuged at 7,700 g for 15 minutes, weighed, and stored at −20° C. for at least 3 hours. The frozen cell pellet was resuspended in 100 mL ice-cold PBS containing 250 μL protease inhibitor cocktail (Sigma Chemical, St. Louis, Mo.) per gram of cells, sonicated at 3× for 1 minute with 15 second breaks. The cells were then centrifuged at 10,000 g for 20 min. The supernatant was mixed with 2 mL of 50% Glutathione Sepharose 4B resin (Pharmacia) on a shaker overnight at 4° C. The supernatant/resin was centrifuged at 1,500 g for 15 min., packed into empty Poly-Prep Chromatography Columns (Bio-Rad, Hercules, Calif.), washed with 30 mL PBS followed by 10 mL Factor Xa buffer (1 mM CaCl₂, 100 mM NaCl, and 50 mM Tris-HCl, pH 8.0). The peptides were cleaved from the column by adding 60 units of Factor Xa (Pharmacia) in 1 mL Factor Xa buffer, incubated overnight at 4° C., and separated by C18 HPLC (Beckman System Gold), using a 2 mL loop and flow rate of 2 mumin with the following program: 10 min. of Buffer A (0.1% TFA/H₂O), 30 min. of gradient to Buffer B (0.1% TFA/ACN), 10 min. of Buffer A, 10 min. of gradient, and 10 min. of Buffer A. Peak fractions (1 mL each) were collected and screened by 10-20% Tricine-SDS gel electrophoresis. Fractions containing the peptides of FIG. 1 were pooled and dried down. Typical yields were several hundred micrograms of free peptides per liter of E. coli culture. Recombinant peptides were shown to have the same activities as their synthetic versions.

### Example 5

### Cyclic AMP SPA

CHO cells expressing the VPAC2 peptide were plated in 96-well plates at 8×10⁴ cells/well and grown at 37° C. for 24 hours in αMEM, nucleosides, glutamine (Gibco/BRL, Rockville, Md.), 5% FBS, 100 μg/mL Pen/Strep, 0.4 mg/mL hygromycin, and 1.5 mg/mL Geneticin (Gibco/BRL). The media was removed and the plates were washed with PBS. The cells were incubated with a peptide (in 10 mM Hepes, 150 mM NaCl, 5 mM KCl, 2.5 mM CaCl₂, 1.2 mM KH₂PO₄, 1.2 mM MgSO₄, 25 mM NaHCO₃ (pH 7.4) with 1% BSA and 100 μM IBMX) for 15 min. at 37° C. Cyclic AMP in the cell extracts was quantitated using the cAMP SPA direct screening assay system (Amersham Pharmacia Biotech Inc., Piscataway, N.J.,). The amount of cAMP present in the lysates was determined following instructions provided with this kit. The amount of cAMP (in pmol) produced at each concentration of peptide was plotted and analyzed by nonlinear regression using Prizm software to determine the EC₅₀ for each peptide.

When a PEG moiety (22 kD or 43 kD) was attached to the C-terminal cysteine of VPAC2 selective VIP mutein peptides (e.g., P5, P12, P212, and P412) potent VPAC2 receptor activation, as measured by increased levels cAMP in cells overexpressing the VPAC2 receptor, was retained. Furthermore, VPAC2 versus VPAC1 selectivity of receptor activation was also retained. The results of the cAMP assay with the representative polypeptides are shown in Table 1. Peptides identified as P5, P12, P212 (P12+22 kD PEG), and P412 (P12+43 kD PEG) are all potent agonists of the VPAC2 receptor, activating the receptor to 100% the maximal level of receptor activation achieved by the endogenous peptide, PACAP-27. Furthermore, the peptides identified as P5, P12, P212, and P412 are selective VPAC2 receptor agonists, possessing very weak agonist activity on VPAC1. PACAP-27 is a potent agonist of both the VPAC1 and VPAC2 receptors. The polypeptides are designed based on VIP sequence, which has been shown to lack activity at PACI (Vaudry, et al., Pharmacol. Rev. 52:269-324, 2000) and polypeptides, P5, P12, P212, and P412 do not possess appreciable activity at PAC1.

TABLE 1
VPAC1 and VPAC2 agonist activity of polypeptides

| | VPAC2 | VPAC1 |
|---|---|---|
| Peptide | EC₅₀ (nM) | EC₅₀ (nM) |
| PACAP-27 | 0.09 | 0.35 |
| P5* | 0.33 | 232.5 |
| P12** | 0.38 | >1000 |
| P212 | 1.32 | >1000 |
| P412 | 4.19 | >1000 |

*P5: HSDAVFTDQYTRLRKQVAAKKYLQSIKQKRY
**P12: HSDAVFTDQYTRLRKQVAAKKYLQSIKQKRYC

### Example 6

Insulin Secretion from Dispersed Rat Islet Cells

Insulin secretion of dispersed rat islets mediated by a number of peptides of the present invention was measured as follows. Islets of Langerhans, isolated from SD rats (200-250 g), were digested using coliagenase. The dispersed islet cells were treated with trypsin, seeded into 96 V-bottom plates, and pelleted. The cells were then cultured overnight in media with or without peptides of this invention. The media was aspirated, and the cells were pre-incubated with Krebs-Ringer-HEPES buffer containing 3 mM glucose for 30 minutes at 37° C. The pre-incubation buffer was removed, and the cells were incubated at 37° C. with Krebs-Ringer-HEPES buffer containing the appropriate glucose concentration (e.g., 8 mM) with or without peptides for an appropriate time. In some studies, an appropriate concentration of GLP-1 was also included. A portion of the supernatant was removed and its insulin content was measured by SPA. The results were expressed as “fold over control” (FOC).

At a concentration of 300 nM, the polypeptide P412 (i.e., peptide P12+43 kD PEG), increased insulin secretion from dispersed islet cells by approximately 1.7-fold. The PEGylated peptides have prolonged activity in vivo to promote insulin secretion, leading to a reduction in blood glucose levels compared to vehicle treated animals following a glucose challenge. The representative PEGylated peptides, P212 and P412, significantly reduced blood glucose levels relative to the vehicle (17%-28% reduction in the glucose AUC) in an IPGTT (Intraperitoneal Glucose Tolerance Test) when the peptides were administered 3 hours prior to the glucose challenge. In addition to the glucose lowering activity of the PEGylated peptides, the ability of the PEGylated peptides to lower blood glucose over a prolonged period of time (e.g., 3 hours) following peptide administration is a clear indication that the PEGylated peptide is present in the circulation at this time point and hence, has prolonged half-life relative to PACAP-27. PACAP-27 has a very short half-life in vivo (<10 min.).

### Example 7

### Measurement of Blood Pressure in Anesthetized Rats

Blood pressure in rats was measured following administration of either non-PEGylated or PEGylated VPAC2 agonist peptides. Blood pressure was measured as follows: Male Wistar rats were anesthetized with pentobarbital (55 mg/kg, i.p.) and the right carotid artery and jugular vein were cannulated. The carotid cannula was connected to the Biopac System (Harvard Apparatus Co., Harvard, Mass.) for continuous monitoring of blood pressure and heart rate. Vehicle or peptide was administered by injection through the jugular vein catheter.

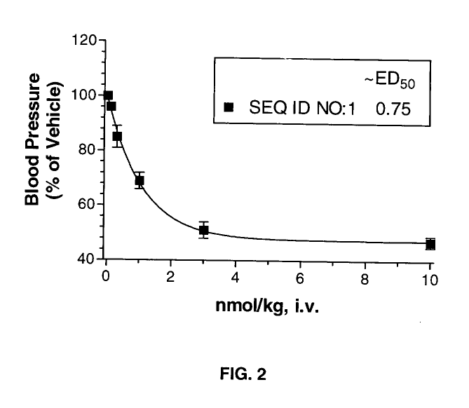

The non-PEGylated peptide, P12, lowers blood pressure dose dependently when administered intravenously (iv) in rats, with and ED₅₀ value of 3 μg/kg (FIG. 2).

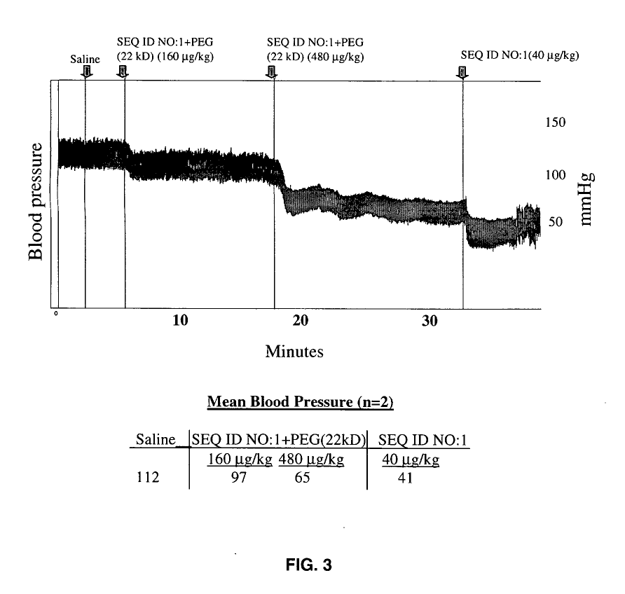

The peptide PEGylated with a linear 22 kD PEG (P212) lowers blood pressure at intravenous doses of >160 μg/kg given as a bolus injection, although the blood pressure lowering effect is less than that of the non-PEGylated peptide (P12) (FIG. 3).

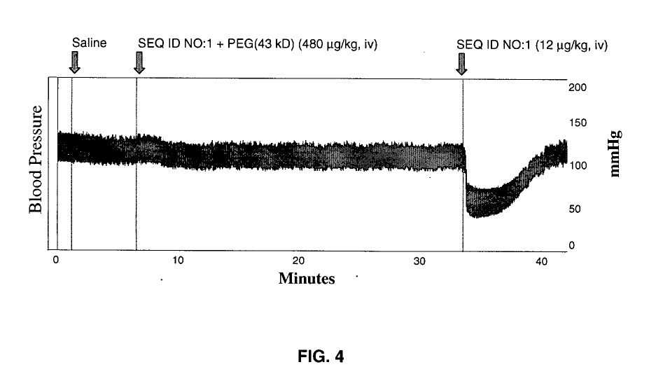

The peptide PEGylated with a branched 43 kD PEG (P412) had no effect on blood pressure at intravenous doses of 1.6 to 480 μg/kg given as a bolus intravenous injection in rats (FIG. 4). The estimated plasma concentration following a 480 μg/kg iv dose of P412 (>4000 nM) is estimated to be >4000-fold the plasma concentration of P412 at the ED₅₀ in the rat IP glucose tolerance test (IPGTT), which is estimated to be <1 nM.

P412 was also administered to two dogs by bolus iv injection in increasing doses of 1, 3, 10, and 30 μg/kg at 1 hour intervals. Blood pressure, heart rate, and cardiovascular parameters were continuously monitored throughout the study. P412 was well tolerated with no effects were observed in any of the parameters measured. Therefore, at systemic exposure levels well above the estimated therapeutic levels, there was no effect on cardiovascular parameters induced by P412 in the dog, a species known to be highly sensitive to cardiovascular effects.

### Example 8

### Measurement of Peptide Effects on Rat Portal Vein Relaxation

It is hypothesized that unlike the highly vascularized pancreatic islets, the 43 kD PEGylated peptide (P412) is unable to efficiently access VPAC2 in the less well vascularized smooth muscle tissue surrounding the blood vessel wall. As a consequence, P412 is unable to promote vascular smooth muscle relaxation which leads to reduced blood pressure. This hypothesis is supported by the results of an isolated rat portal vein tissue bath study.

The portal vein from Wistar rats was incubated at 32° C. in Krebs (pH 7.4) in a tissue bath (10 ml) in the presence of vehicle (PBS, pH 7), or peptides at the indicated concentrations for 10 minutes. Isometric changes in vessel tension were assessed and are reported relative to the response to VIP.

In this study, it was shown that P12 (i.e., non-PEGylated peptide) caused portal vein relaxation with an ED₅₀=0.3 nM (similar to its EC₅₀ of 0.4 nM in the cellular cAMP assay). On the other hand, the 43 kD PEGylated peptide failed to cause any portal vein relaxation at the highest dose tested (30 nM), which is >7-fold above its EC₅₀ (4.2 nM) in the cellular cAMP assay.

TABLE 2
Peptide Effects Relative to VIP on Rat Portal vein Relaxation

| | Concentration | Agonist Activity |
|---|---|---|
| Peptide | (nM) | (% VIP) |
| P12 | 3 | 87 |
| (non-PEGylated) | 1 | 70 |
| | 0.3 | 52 |
| P12 + | 30 | 0 |
| 43 kD PEG | 10 | 0 |
| | 3 | 0 |

### Example 9

### Effect of PEGylated Peptides on Intraperitoneal Glucose Tolerance in Rats

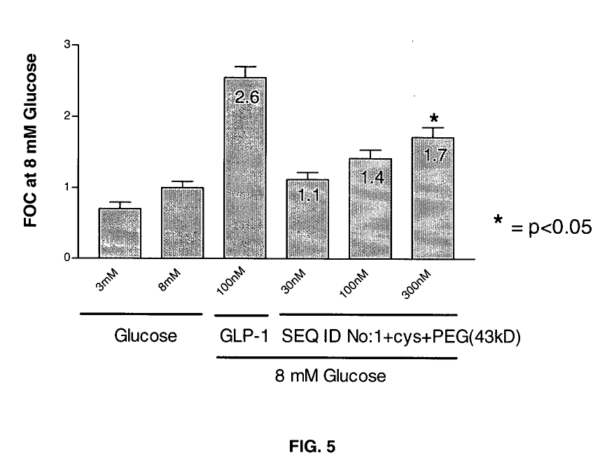

The in vivo activity of the PEGylated peptides of this invention when administered subcutaneously was examined in rats. Rats fasted overnight were given a subcutaneous injection of control or PEGylated peptide (1-100 μg/kg). Three hours later, basal blood glucose was measured, and the rats were given 2 g/kg of glucose intraperitoneally. Blood glucose was measured again after 15, 30, and 60 min. The representative PEGylated peptide of this invention significantly reduced blood glucose levels relative to the vehicle following the IPGTT (Intraperitoneal Glucose Tolerance Test), with 17%-28% reduction in the glucose AUC (FIG. 5). This demonstrates that the PEGylated peptide has prolonged glucose lowering activity in vivo. In addition to the glucose lowering activity of the PEGylated peptides of the present invention, it also indicates prolonged peptide half-life in vivo. PACAP-27 has a very short half-life in vivo (<10 min.). The ability of the PEGylated peptides of the invention to lower blood glucose 3 hours following peptide administration is a clear indication that the peptide is present in the circulation at this time point and hence, has prolonged half-life relative to PACAP-27.

All publications and patents mentioned in the above specification are incorporated herein by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

1. A polypeptide selected from the group consisting of SEQ ID NOs: 1-153 and functionally equivalent fragments, derivatives, and variants thereof.

2. The polypeptide of claim 1, wherein said polypeptide is linked to a polyethylene glycol polymer.

3. The polypeptide of claim 2, wherein said polyethylene glycol has a molecular weight of at least 22 kD.

4. The polypeptide of claim 3, wherein said polyethylene glycol is branched.

5. The polypeptide of claim 1, wherein said polypeptide is acetylated.

6. A polynucleotide encoding a polypeptide sequence of claim 1, or a degenerate variant thereof.

7. A vector comprising a polynucleotide of claim 6.

8. A host cell comprising a vector of claim 7.

9. A method for producing a polypeptide comprising:
a) culturing the host cell of claim 8 under conditions suitable for the expression of said polypeptide; and
b) recovering the polypeptide from the host cell culture.

10. A method for reducing or inhibiting blood pressure side effects of a VPAC2 receptor agonist comprising the step of linking a polyethylene glycol polymer to said VPAC2 receptor agonist.

11. The method of claim 10, wherein said polyethylene glycol has a molecular weight of at least 22 kD.

12. The method of claim 11, wherein said polyethylene glycol is branched.

13. The method of claim 10, wherein said GLP-1 receptor agonist is selected from the group consisting of SEQ ID NOs: 1-153 and functionally equivalent fragments, derivatives, and variants thereof.

14. A pharmaceutical composition comprising a therapeutically effective amount of a polypeptide of any one of claims 1 to 5, or functionally equivalent fragments, derivatives, and variants thereof, in combination with a pharmaceutically acceptable carrier.

15. A pharmaceutical composition comprising a therapeutically effective amount of a polypeptide of any one of claims 1 to 5, or functionally equivalent fragments, derivatives, and variants thereof, in combination with a pharmaceutically acceptable carrier and one or more additional pharmaceutical agents.

16. The pharmaceutical composition of claim 15, wherein said pharmaceutical agent is selected from the group consisting of PPAR ligands, insulin secretagogues, sulfonylurea drugs, α-glucosidase inhibitors, insulin sensitizers, hepatic glucose output lowering compounds, insulin and insulin derivatives, biguanides, protein tyrosine phosphatase-1B, dipeptidyl peptidase IV, 11beta-HSD inhibitors, anti-obesity drugs, HMG-CoA reductase inhibitors, nicotinic acid, lipid lowering drugs, ACAT inhibitors, bile acid sequestrants, bile acid reuptake inhibitors, microsomal triglyceride transport inhibitors, fibric acid derivatives, β-blockers, ACE inhibitors, calcium channel blockers, diuretics, renin inhibitors, AT-1 receptor antagonists, ET receptor antagonists, neutral endopeptidase inhibitors, vasopepsidase inhibitors, and nitrates.

17. A method of treating diabetes in a subject comprising the step of administering to a subject in need thereof a therapeutically effective amount of a polypeptide of any one of claims 1 to 5 or pharmaceutical composition of claim 14.

18. The method of claim 17, wherein said diabetes is selected from the group consisting of type 1 diabetes, type 2 diabetes, maturity-onset diabetes of the young, latent autoimmune diabetes adult, and gestational diabetes.

19. The method of claim 17, wherein said polypeptide is administered in combination with one or more pharmaceutical agents.

20. A method of treating Syndrome X comprising the step of administering to a subject in need thereof a therapeutically effective amount of a polypeptide of any one of claims 1 to 5 or pharmaceutical composition of claim 14.

21. A method of treating diabetes-related disorders comprising the step of administering to a subject in need thereof a therapeutically effective amount of a polypeptide of any one of claims 1 to 5 or pharmaceutical composition of claim 14.

22. The method of claim 21, wherein said diabetes-related disorder is selected from the group consisting of hyperglycemia, hyperinsulinemia, impaired glucose tolerance, impaired fasting glucose, dyslipidemia, hypertriglyceridemia, and insulin resistance.

23. A method of treating or preventing secondary causes of diabetes comprising the step of administering to a subject in need thereof a therapeutically effective amount of a polypeptide of any one of claims 1 to 5 or pharmaceutical composition of claim 14.

24. A method of treating diabetes comprising the step of administering to a subject in need thereof a therapeutically effective amount of a polypeptide of any one of claims 1 to 5 or pharmaceutical composition of claim 14 in combination with one or more additional pharmaceutical agents.

25. The method of claim 24, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, and anti-obesity agents.

26. The method of claim 24, wherein said diabetes is selected from the group consisting of type 1 diabetes, type 2 diabetes, maturity-onset diabetes of the young, latent autoimmune diabetes adult, and gestational diabetes.

27. A method of treating Syndrome X comprising the step of administering to a subject in need thereof a therapeutically effective amount of a polypeptide of any one of claims 1 to 5 or pharmaceutical composition of claim 14 in combination with one or more additional pharmaceutical agents.

28. The method of claim 27, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, and anti-obesity agents.

29. A method of treating diabetes-related disorders comprising the step of administering to a subject in need thereof a therapeutically effective amount of a polypeptide of any one of claims 1 to 5 or pharmaceutical composition of claim 14 in combination with one or more additional pharmaceutical agents.

30. The method of claim 29, wherein said diabetes-related disorder is selected from the group consisting of hyperglycemia, hyperinsulinemia, impaired glucose tolerance, impaired fasting glucose, dyslipidemia, hypertriglyceridemia, and insulin resistance.

31. The method of claim 29, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, and anti-obesity agents.

32. A method of treating or preventing secondary causes of diabetes comprising the step of administering a subject in need thereof a therapeutically effective amount of a polypeptide of any one of claims 1 to 5 or pharmaceutical composition of claim 14 in combination with one or more additional pharmaceutical agents.

33. The method of claim 32, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, and anti-obesity agents

34. A method of treating diabetes, Syndrome X, diabetes-related disorders or secondary causes of diabetes comprising the step of administering to a subject in need thereof a therapeutically effective amount of a polypeptide of any one of claims 1 to 5 or pharmaceutical composition of claim 14 in combination with one or more additional pharmaceutical agents selected from the group consisting of HMG-CoA reductase inhibitors, nicotinic acid, lipid lowering drugs, ACAT inhibitors, bile acid sequestrants, bile acid reuptake inhibitors, microsomal triglyceride transport inhibitors, fibric acid derivatives, β-blockers, ACE inhibitors, calcium channel blockers, diuretics, renin inhibitors, AT-1 receptor antagonists, ET receptor antagonists, neutral endopeptidase inhibitors, vasopepsidase inhibitors, and nitrates.

35. The method of claim 34, wherein said diabetes-related disorder is selected from the group consisting of hyperglycemia, hyperinsulinemia, impaired glucose tolerance, impaired fasting glucose, dyslipidemia, hypertriglyceridemia, and insulin resistance.

36. The method of any one of claims 24 to 35, wherein the polypeptide and one or more pharmaceutical agents are administered as a single pharmaceutical dosage formulation.

37. Polypeptides according to any one of claims 1 to 5 for the treatment and/or prophylaxis of diabetes.

38. Medicament containing at least one polypeptide according to any one of claims 1 to 5 in combination with at least one pharmaceutically acceptable, pharmaceutically safe carrier or excipient.

39. Use of polypeptides according to any one of claims 1 to 5 for manufacturing a medicament for the treatment and/or prophylaxis of diabetes.

40. Medicament according to claim 38 for the treatment and/or prophylaxis of diabetes.
